(19) United States
(12) Patent Application Publication
(10) Pub. No.: US 2005/0272726 A1
(43) Pub. Date: Dec. 8, 2005

(54) Novel medicaments for the treatment of respiratory diseases

(75) Inventors: Ingo Konetzki, Warthausen (DE); Thierry Bouyssou, Mietingen (DE); Philipp Lustenberger, Warthausen (DE); Frank Buettner, Attenweiler (DE); Andreas Schnapp, Biberach (DE); Christoph Hoenke, Ingelheim (DE); Sabine Pestel, Attenweiler (DE); Klaus Rudolf, Warthausen (DE); Hermann Schollenberger, Ingelheim (DE); Kurt Schromm, Ingelheim (DE); Claudia Heine, Biberach (DE)

Correspondence Address: MICHAEL P. MORRIS;BOEHRINGER INGELHEIM CORPORATION, 900 RIDGEBURY ROAD, P. O. BOX 368, RIDGEFIELD, CT, 06877-0368, US

(73) Assignee: Boehringer Ingelheim International GmbH, Ingelheim (DE)

(21) Appl. No.: 11/109,030

(22) Filed: Apr. 19, 2005

Related U.S. Application Data
(60) Provisional application No. 60/578,569, filed on Jun. 10, 2004.

(30) Foreign Application Priority Data
Apr. 22, 2004 (DE) 10 2004 019 539.0

(51) Int. Cl. A61K 31/538

(52) U.S. Cl. 514/230.5

(57) ABSTRACT

The present invention relates to the use of the compounds of general formula 1 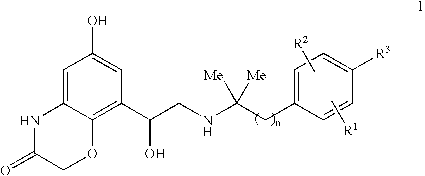
wherein the groups R¹, R² and R³ may have the meanings given in the claims and in the specification, for preparing a pharmaceutical composition for the treatment of respiratory complaints.

This application claims priority benefit under 35 USC 119(e) from U.S. Provisional Application 60/578,569, filed Jun. 10, 2004 and from German Application DE 10 2004 019 539.0, filed Apr. 22, 2004.

The present invention relates to the use of the compounds of general formula 1 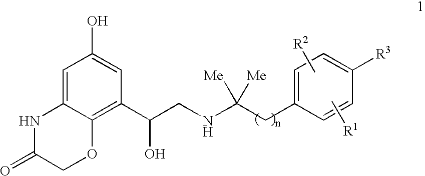
wherein the groups R¹, R² and R³ may have the meanings given in the claims and in the specification, for preparing a pharmaceutical composition for the treatment of respiratory complaints.

### BACKGROUND TO THE INVENTION

Betamimetics (β-adrenergic substances) are known from the prior art. For example reference may be made in this respect to the disclosure of U.S. Pat. No. 4,460,581, which proposes betamimetics for the treatment of a range of diseases.

For drug treatment of diseases it is often desirable to prepare medicaments with a longer duration of activity. As a rule, this ensures that the concentration of the active substance in the body needed to achieve the therapeutic effect is guaranteed for a longer period without the need to re-administer the drug at frequent intervals. Moreover, giving an active substance at longer time intervals contributes to the well-being of the patient to a high degree. It is particularly desirable to prepare a pharmaceutical composition which can be used therapeutically by administration once a day (single dose). The use of a drug once a day has the advantage that the patient can become accustomed relatively quickly to regularly taking the drug at certain times of the day.

The aim of the present invention is therefore to provide betamimetics which on the one hand confer a therapeutic benefit in the treatment of respiratory complaints and are also characterised by a longer duration of activity and can thus be used to prepare pharmaceutical compositions with a longer duration of activity. A particular aim of the invention is to prepare betamimetics which, by virtue of their long-lasting effect, can be used to prepare a drug for administration once a day for treating asthma. A further objective of the invention, apart from those mentioned above, is to prepare new betamimetics which are not only exceptionally potent but are also characterised by a high degree of selectivity with respect to the β-adrenoceptor.

### DETAILED DESCRIPTION OF THE INVENTION

It has been found that, surprisingly, the above-mentioned problems are solved by compounds of general formula 1.

Accordingly, the present invention relates to the use of one or more, preferably one, compound of general formula 1 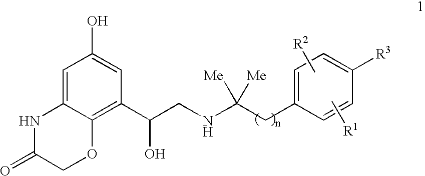
wherein
n denotes 1 or 2, preferably 1;
R¹ denotes hydrogen, halogen, C₁-C₄-alkyl or —O—C₁-C₄-alkyl;
R² denotes hydrogen, halogen, C₁-C₄-alkyl or —O—C₁-C₄-alkyl;
R³ denotes C₁-C₄-alkyl, OH, halogen, —O—C₁-C₄-alkyl, —O—C₁-C₄-alkylene-COOH, —O—C₁-C₄-alkylene-CO—O—C₁-C₄-alkyl,
   with the proviso that if R¹ and R² each denote ortho-methyl, R³ cannot simultaneously be OH, for preparing a pharmaceutical composition for the treatment of respiratory complaints selected from the group comprising obstructive pulmonary diseases of various origins, pulmonary emphysema of various origins, restrictive pulmonary diseases, interstitial pulmonary diseases, cystic fibrosis, bronchitis of various origins, bronchiectasis, ARDS (adult respiratory distress syndrome) and all forms of pulmonary oedema.

Preferably, the compounds of general formula 1 used as specified above are those wherein
n denotes 1 or 2, preferably 1;
R¹ denotes hydrogen, fluorine, chlorine, methyl or methoxy;
R² denotes hydrogen, fluorine, chlorine, methyl or methoxy;
R³ denotes C₁-C₄-alkyl, OH, fluorine, chlorine, bromine, —O—C₁-C₄-alkyl, —O—C₁-C₄-alkylene-COOH, —O—C₁-C₄-alkylene-CO—O—C₁-C₄-alkyl,
   with the proviso that if R¹ and R² each denote ortho-methyl, R³ cannot simultaneously be OH.

Preferably, the compounds of general formula 1 used as specified above are those wherein
n denotes 1;
R¹ denotes hydrogen or C₁-C₄-alkyl;
R² denotes hydrogen or C₁-C₄-alkyl;
R³ denotes C₁-C₄-alkyl, OH, —O—C₁-C₄-alkyl, —O—C₁-C₄-alkylene-COOH or —O—C₁-C₄— alkylene-CO—O—C₁-C₄-alkyl,
   with the proviso that if R¹ and R² each denote ortho-methyl, R³ cannot simultaneously be OH.

Preferably, the compounds of general formula 1 used as specified above are those wherein
n denotes 1;
R¹ denotes hydrogen, methyl or ethyl;
R² denotes hydrogen, methyl or ethyl;
R³ denotes methyl, ethyl, OH, methoxy, ethoxy, —O—CH₂—COOH, —O—CH₂—COOmethyl or —O—CH₂—COOethyl,
   with the proviso that if R¹ and R² each denote ortho-methyl, R³ cannot simultaneously be OH.

Preferably, the compounds of general formula 1 used as specified above are those wherein
n denotes 1;
R¹ denotes hydrogen or methyl;
R² denotes hydrogen or methyl;
R³ denotes methyl, OH, methoxy, —O—CH₂—COOH or —O—CH₂—COOethyl,
   with the proviso that if R¹ and R² each denote ortho-methyl, R³ cannot simultaneously be OH.

Preferably, according to the invention, the compounds of general formula 1 used as specified above are those wherein
R³ denotes methoxy, ethoxy, —O—CH₂—COOH, —O—CH₂—COOmethyl or —O—CH₂—COOethyl,
   and R¹, R² and n may have the meanings given above.

The present invention further relates to the above-mentioned use of compounds of general formula 1,

wherein

n denotes 1;
R¹ denotes halogen, C₁-C₄-alkyl or —O—C₁-C₄-alkyl;
R² denotes halogen, C₁-C₄-alkyl or —O—C₁-C₄-alkyl;
R³ denotes halogen, C₁-C₄-alkyl or —O—C₁-C₄-alkyl.

The present invention further relates to the above-mentioned use of compounds of general formula 1,

wherein

n denotes 1;
R¹ denotes fluorine, chlorine, methyl or methoxy;
R² denotes fluorine, chlorine, methyl or methoxy;
R³ denotes fluorine, chlorine, methyl or methoxy.

According to another preferred aspect of the present invention the compounds of general formula 1 used as specified above are those wherein
n denotes 1;
R¹ denotes hydrogen;
R² denotes hydrogen, fluorine, chlorine or methyl;
R³ denotes methyl, ethyl, iso-propyl, tert.-butyl, OH, fluorine, chlorine, bromine, methoxy, ethoxy, —O—CH₂—COOH, —O—CH₂—CH₂-COOH, —O—CH₂—CH₂—CH₂—COOH, —O—CH₂—COOmethyl, —O—CH₂—COOethyl, —O—CH₂—CH₂—COOmethyl, —O—CH₂—CH₂—COOethyl, —O—CH₂—CH₂—CH₂—COOmethyl or —O—CH₂—CH₂—CH₂—COOethyl.

Particularly preferably, the compounds of general formula 1 used as specified above are those wherein
n denotes 1;
R¹ denotes hydrogen;
R² denotes hydrogen, fluorine, chlorine or methyl;
R³ denotes OH, fluorine, chlorine, methyl, methoxy, ethoxy or —O—CH₂—COOH.

Moreover it is particularly preferable according to the invention if the compounds of general formula 1 according to the invention used as specified above are those wherein
n denotes 1;
R¹ denotes hydrogen;
R² denotes halogen, C₁-C₄-alkyl or —O—C₁-C₄-alkyl, preferably fluorine, chlorine, methoxy or methyl;
R³ denotes halogen, C₁-C₄-alkyl or —O—C₁-C₄-alkyl, preferably fluorine, chlorine, methoxy or methyl.

In another preferred aspect of the present invention the compounds of general formula 1 according to the invention used as specified above are those wherein n=1, R¹ and R² denote hydrogen and the group R³ may have the meanings given above.

In another preferred aspect of the present invention the compounds of general formula 1 according to the invention used as specified above are those wherein
R¹ and R² denote hydrogen;
R³ denotes methyl, ethyl, iso-propyl, tert.-butyl, OH, fluorine, chlorine, bromine, methoxy, ethoxy, —O—CH₂—COOH, —O—CH₂—CH₂—COOH, —O—CH₂—CH₂—CH₂—COOH, —O—CH₂—COOmethyl, —O—CH₂—COOethyl, —O—CH₂—CH₂—COOmethyl, —O—CH₂—CH₂—COOethyl, —O—CH₂—CH₂—CH₂—COOmethyl, —O—CH₂—CH₂—CH₂—COOethyl.

Particularly preferably the compounds of general formula 1 according to the invention used as specified above are those wherein
n denotes 1;
R¹ and R² denote hydrogen;
R³ denotes OH, fluorine, chlorine, methoxy, ethoxy, —O—CH₂—COOH, preferably OH, fluorine, chlorine, ethoxy or methoxy.

Particularly preferably the compounds of general formula 1 according to the invention used as specified above are those wherein
n denotes 1;
R¹ and R² denote hydrogen;
R³ denote fluorine, chlorine, methoxy or ethoxy.

The present invention further relates to the above-mentioned use of compounds of general formula 1, wherein
n denotes 1;
R¹ denotes hydrogen, halogen, C₁-C₄-alkyl or —O—C₁-C₄-alkyl;
R² denotes hydrogen, halogen, C₁-C₄-alkyl or —O—C₁-C₄-alkyl;
R³ denotes hydrogen.

Preferably, the compounds of general formula 1 used as specified above are those wherein
n denotes 1;
R¹ denotes hydrogen, fluorine, chlorine, methyl or methoxy;
R² denotes hydrogen, fluorine, chlorine, methyl or methoxy;
R³ denotes hydrogen.

The present invention further relates to the above-mentioned use of compounds of general formula 1, wherein
n denotes 1;
R¹ denotes fluorine, chlorine, methyl or methoxy;
R² denotes fluorine, chlorine, methyl or methoxy;
R³ denotes hydrogen.

In the compounds of formula 1 the groups R¹ and R², if they do not represent hydrogen, may each be arranged in the ortho or meta position relative to the bond to the benzylic “—CH₂” group. If none of the groups R¹ and R² denotes hydrogen, preferred compounds of formula 1 for the use according to the invention are those wherein either both groups R¹ and R² are ortho or both groups R¹ and R² are in the meta configuration, while compounds in which both groups R¹ and R² are in the ortho-configuration are of particular importance. In the compounds of formula 1 wherein one of the groups R¹ and R² does not denote hydrogen, this group may be in the ortho or meta position relative to the bond to the benzylic “—CH₂” group. In this case those compounds of formula 1 wherein the group R¹ or R² which does not denote hydrogen is in the ortho configuration are particularly preferred for use according to the invention.

Particularly preferably, one or more of the following compounds of general formula 1 are used for the purpose described above:
6-hydroxy-8-{1-hydroxy-2-[2-(4-methoxy-phenyl)-1,1-dimethyl-ethylamino]-ethyl}-4H-benzo[1,4]oxazin-3-one;
6-hydroxy-8-{1-hydroxy-2-[2-(4-ethyl-phenoxy-acetate)-1,1-dimethyl-ethylamino]-ethyl}-4H-benzo[1,4]oxazin-3-one;
6-hydroxy-8-{1-hydroxy-2-[2-(4-phenoxy-acetic acid)-1,1-dimethyl-ethylamino]-ethyl}-4H-benzo[1,4]oxazin-3-one;
8-{2-[1,1-dimethyl-2-(2,4,6-trimethylphenyl)-ethylamino]-1-hydroxy-ethyl}-6-hydroxy-4H-benzo[1,4]oxazin-3-one;
6-hydroxy-8-{1-hydroxy-2-[2-(4-hydroxy-phenyl)-1,1-dimethyl-ethylamino]-ethyl}-4H-benzo[1,4]oxazin-3-one;
6-hydroxy-8-{1-hydroxy-2-[2-(4-isopropyl-phenyl)-1,1-dimethyl-ethylamino]-ethyl}-4H-benzo[1,4]oxazin-3-one;
8-{2-[2-(4-ethyl-phenyl)-1,1-dimethyl-ethylamino]-1-hydroxy-ethyl}-6-hydroxy-4H-benzo[1,4]oxazin-3-one;
8-{2-[2-(4-fluoro-3-methyl-phenyl)-1,1-dimethyl-ethylamino]-1-hydroxy-ethyl}-6-hydroxy-4H-benzo[1,4]oxazin-3-one;
8-{2-[2-(4-fluoro-2-methyl-phenyl)-1,1-dimethyl-ethylamino]-1-hydroxy-ethyl}-6-hydroxy-4H-benzo[1,4]oxazin-3-one;
8-{2-[2-(2,4-difluoro-phenyl)-1,1-dimethyl-ethylamino]-1-hydroxy-ethyl}-6-hydroxy-4H-benzo[1,4]oxazin-3-one;
8-{2-[2-(3,5-difluoro-phenyl)-1,1-dimethyl-ethylamino]-1-hydroxy-ethyl}-6-hydroxy-4H-benzo[1,4]oxazin-3-one;
8-{2-[2-(4-ethoxy-phenyl)-1,1-dimethyl-ethylamino]-1-hydroxy-ethyl}-6-hydroxy-4H-benzo[1,4]oxazin-3-one;
8-{2-[2-(3,5-dimethyl-phenyl)-1,1-dimethyl-ethylamino]-1-hydroxy-ethyl}-6-hydroxy-4H-benzo[1,4]oxazin-3-one;
4-(4-{2-[2-hydroxy-2-(6-hydroxy-3-oxo-3,4-dihydro-2H-benzo[1,4]oxazin-8-yl)-ethylamino]-2-methyl-propyl}-phenoxy)-butyric acid;
8-{2-[2-(3,4-difluoro-phenyl)-1,1-dimethyl-ethylamino]-1-hydroxy-ethyl}-6-hydroxy-4H-benzo[1,4]oxazin-3-one;
8-{2-[2-(2-chloro-4-fluoro-phenyl)-1,1-dimethyl-ethylamino]-1-hydroxy-ethyl}-6-hydroxy-4H-benzo[1,4]oxazin-3-one;
8-{2-[2-(4-chloro-phenyl)-1,1-dimethyl-ethylamino]-1-hydroxy-ethyl}-6-hydroxy-4H-benzo[1,4]oxazin-3-one;
8-{2-[2-(4-bromo-phenyl)-1,1-dimethyl-ethylamino]-1-hydroxy-ethyl}-6-hydroxy-4H-benzo[1,4]oxazin-3-one;
8-{2-[2-(4-fluoro-phenyl)-1,1-dimethyl-ethylamino]-1-hydroxy-ethyl}-6-hydroxy-4H-benzo[1,4]oxazin-3-one;
8-{2-[2-(4-fluoro-3-methoxy-phenyl)-1,1-dimethyl-ethylamino]-1-hydroxy-ethyl}-6-hydroxy-4H-benzo[1,4]oxazin-3-one;
8-{2-[2-(4-fluoro-2,6-dimethyl-phenyl)-1,1-dimethyl-ethylamino]-1-hydroxy-ethyl}-6-hydroxy-4H-benzo[1,4]oxazin-3-one;
8-{2-[2-(4-chloro-2-methyl-phenyl)-1,1-dimethyl-ethylamino]-1-hydroxy-ethyl}-6-hydroxy4H-benzo[1,4]oxazin-3-one;
8-{2-[2-(4-chloro-3-fluoro-phenyl)-1,1-dimethyl-ethylamino]-1-hydroxy-ethyl}-6-hydroxy-4H-benzo[1,4]oxazin-3-one;
8-{2-[2-(4-chloro-2-fluoro-phenyl)-1,1-dimethyl-ethylamino]-1-hydroxy-ethyl}-6-hydroxy-4H-benzo[1,4]oxazin-3-one;
8-{2-[2-(3-chloro-4-fluoro-phenyl)-1,1-dimethyl-ethylamino]-1-hydroxy-ethyl}-6-hydroxy-4H-benzo[1,4]oxazin-3-one;
8-{2-[2-(2,6-difluoro-4-methoxy-phenyl)-1,1-dimethyl-ethylamino]-1-hydroxy-ethyl}-6-hydroxy-4H-benzo[1,4]oxazin-3-one;
8-{2-[2-(2,5-difluoro-4-methoxy-phenyl)-1,1-dimethyl-ethylamino]-1-hydroxy-ethyl}-6-hydroxy-4H-benzo[1,4]oxazin-3-one;
8-{2-[2-(4-fluoro-3,5-dimethyl-phenyl)-1,1-dimethyl-ethylamino]-1-hydroxy-ethyl}-6-hydroxy-4H-benzo[1,4]oxazin-3-one;
8-{2-[2-(3,5-dichloro-phenyl)-1,1-dimethyl-ethylamino]-1-hydroxy-ethyl}-6-hydroxy-4H-benzo[1,4]oxazin-3-one;
8-{2-[2-(4-chloro-3-methyl-phenyl)-1,1-dimethyl-ethylamino]-1-hydroxy-ethyl}-6-hydroxy-4H-benzo[1,4]oxazin-3-one;
8-{2-[2-(3,4,5-trifluoro-phenyl)-1,1-dimethyl-ethylamino]-1-hydroxy-ethyl}-6-hydroxy-4H-benzo[1,4]oxazin-3-one;
8-{2-[2-(3-methyl-phenyl)-1,1-dimethyl-ethylamino]-1-hydroxy-ethyl}-6-hydroxy-4H-benzo[1,4]oxazin-3-one and
8-{2-[2-(3,4-dichloro-phenyl)-1,1-dimethyl-ethylamino]-1-hydroxy-ethyl}-6-hydroxy-4H-benzo[1,4]oxazin-3-one.

In another aspect the present invention relates to the above-mentioned use of the compounds of formula 1 in the form of the individual optical isomers, or mixtures of the individual enantiomers or racemates. It is particularly preferable to use the compounds of formula 1 for the purpose described above in the form of the enantiomerically pure compounds, while the use of the R-enantiomers of the compounds of formula 1 is of exceptional importance according to the invention.

In another aspect the present invention relates to the above-mentioned use of the compounds of formula 1 in the form of the acid addition salts with pharmacologically acceptable acids as well as optionally in the form of the solvates and/or hydrates.

The compounds of general formula 1 are preferably used for preparing a pharmaceutical composition for the treatment of obstructive pulmonary diseases selected from among bronchial asthma, paediatric asthma, severe asthma, acute asthma attacks and chronic bronchitis, while it is particularly preferable according to the invention to use them for preparing a pharmaceutical composition for the treatment of bronchial asthma.

It is also preferable to use the compounds of general formula 1 for preparing a pharmaceutical composition for the treatment of pulmonary emphysema which has its origins in COPD (chronic obstructive pulmonary disease) or α1-proteinase inhibitor deficiency.

It is also preferable to use the compounds of general formula 1 for preparing a pharmaceutical composition for the treatment of restrictive pulmonary diseases selected from among allergic alveolitis, restrictive pulmonary diseases triggered by work-related noxious substances, such as asbestosis or silicosis, and restriction caused by lung tumours, such as for example lymphangiosis carcinomatosa, bronchoalveolar carcinoma and lymphomas.

It is also preferable to use the compounds of general formula 1 for preparing a pharmaceutical composition for the treatment of interstitial pulmonary diseases selected from among pneumonia caused by infections, such as for example infection by viruses, bacteria, fungi, protozoa, helminths or other pathogens, pneumonitis caused by various factors, such as for example aspiration and left heart insufficiency, radiation-induced pneumonitis or fibrosis, collagenoses, such as for example lupus erythematodes, systemic sclerodermy or sarcoidosis, granulomatoses, such as for example Boeck's disease, idiopathic interstitial pneumonia or idiopathic pulmonary fibrosis (IPF).

It is also preferable to use the compounds of general formula 1 for preparing a pharmaceutical composition for the treatment of cystic fibrosis or mucoviscidosis.

It is also preferable to use the compounds of general formula 1 for preparing a pharmaceutical composition for the treatment of bronchitis, such as for example bronchitis caused by bacterial or viral infection, allergic bronchitis and toxic bronchitis.

It is also preferable to use the compounds of general formula 1 for preparing a pharmaceutical composition for the treatment of bronchiectasis.

It is also preferable to use the compounds of general formula 1 for preparing a pharmaceutical composition for the treatment of ARDS (adult respiratory distress syndrome).

It is also preferable to use the compounds of general formula 1 for preparing a pharmaceutical composition for the treatment of pulmonary oedema, for example toxic pulmonary oedema after aspiration or inhalation of toxic substances and foreign substances.

It is particularly preferable to use the compounds detailed above for preparing a pharmaceutical composition for the treatment of asthma or COPD. Also of particular importance is the above-mentioned use of compounds of formula 1 for preparing a pharmaceutical composition for once-a-day treatment of inflammatory and obstructive respiratory complaints, particularly for the once-a-day treatment of asthma.

The present invention also relates to a process for the treatment of the above-mentioned diseases, characterised in that one or more of the above-mentioned compounds of general formula 1 are administered in therapeutically effective amounts. The present invention further relates to processes for the treatment of Asthma, characterised in that one or more of the above-mentioned compounds of general formula 1 are administered once a day in therapeutically effective amounts.

By acid addition salts with pharmacologically acceptable acids are meant for example salts selected from the group comprising the hydrochloride, hydrobromide, hydroiodide, hydrosulphate, hydrophosphate, hydromethanesulphonate, hydronitrate, hydromaleate, hydroacetate, hydrobenzoate, hydrocitrate, hydrofumarate, hydrotartrate, hydrooxalate, hydrosuccinate, hydrobenzoate and hydro-p-toluenesulphonate, preferably the hydrochloride, hydrobromide, hydrosulphate, hydrophosphate, hydrofumarate and hydromethanesulphonate.

Of the above-mentioned acid addition salts, the salts of hydrochloric acid, methanesulphonic acid, benzoic acid and acetic acid are particularly preferred according to the invention.

For use according to the invention the compounds of general formula 1 may optionally be used in the form of the individual optischen isomers, mixtures of the individual enantiomers or racemates. If the compounds are used in enantiomerically pure form, the R-enantiomers are preferably used.

Unless otherwise stated, the alkyl groups are straight-chained or branched alkyl groups having 1 to 4 carbon atoms. The following are mentioned by way of example: methyl, ethyl, propyl or butyl. In some cases the abbreviations Me, Et, Prop or Bu are used to denote the groups methyl, ethyl, propyl or butyl. Unless otherwise stated, the definitions propyl and butyl include all the possible isomeric forms of the groups in question. Thus, for example, propyl includes n-propyl and iso-propyl, butyl includes iso-butyl, sec.butyl and tert.-butyl, etc.

Unless otherwise stated, the alkylene groups are branched and unbranched double-bonded alkyl bridges having 1 to 4 carbon atoms. The following are mentioned by way of example: methylene, ethylene, n-propylene or n-butylene.

Unless otherwise stated, the term alkyloxy groups (or —O-alkyl groups) denotes branched and unbranched alkyl groups having 1 to 4 carbon atoms which are linked via an oxygen atom. Examples of these include: methyloxy, ethyloxy, propyloxy or butyloxy. The abbreviations MeO—, EtO—, PropO— or BuO— are used in some cases to denote the groups methyloxy, ethyloxy, propyloxy or butyloxy. Unless otherwise stated, the definitions propyloxy and butyloxy include all possible isomeric forms of the groups in question. Thus, for example, propyloxy includes n-propyloxy and iso-propyloxy, butyloxy includes iso-butyloxy, sec.butyloxy and tert.-butyloxy, etc. In some cases, within the scope of the present invention, the term alkoxy is used instead of the term alkyloxy. Accordingly, the terms methoxy, ethoxy, propoxy or butoxy may also be used to denote the groups methyloxy, ethyloxy, propyloxy or butyloxy.

halogen within the scope of the present invention denotes fluorine, chlorine, bromine or iodine. Unless stated otherwise, fluorine, chlorine and bromine are the preferred halogens.

The compounds according to the invention may be prepared analogously to methods already known from the prior art. Suitable methods of preparation are known for example from U.S. Pat. No. 4,460,581, to the entire contents of which reference is made at this point.

The examples of synthesis described below serve to illustrate new compounds according to the invention in more detail. However, they are intended only as examples of procedures to illustrate the invention without restricting it to the subject matter described in an exemplifying capacity hereinafter.

### EXAMPLE 1

### 6-hydroxy-8-{1-hydroxy-2-[2-(4-methoxy-phenyl)-1,1-dimethyl-ethylamino]-ethyl}-4H-benzo[1,4]oxazin-3-one

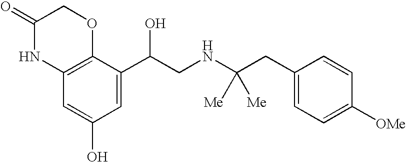

### a) 8-{2-[1,1-dimethyl-2-(4-methoxy-phenyl)-ethylamino]-1-hydroxy-ethyl}-6-benzyloxy-4H-benzo[1,4]oxazin-3-one

7.5 g (6-benzyloxy-4H-benzo[1,4]oxazin-3-one)-glyoxalhydrate are added at 70° C. to a solution of 3.6 g 1,1-dimethyl-2-(4-methoxyphenyl)-ethylamine in 100 mL ethanol and the mixture is stirred for 15 minutes. Then within 30 minutes at 10 to 20° C. 1 g sodium borohydride is added. The mixture is stirred for one hour, combined with 10 mL acetone and stirred for a further 30 minutes. The reaction mixture is diluted with 150 mL ethyl acetate, washed with water, dried with sodium sulphate and evaporated down. The residue is dissolved in 50 mL methanol and 100 mL ethyl acetate and acidified with conc. hydrochloric acid. After the addition of 100 mL diethyl ether the product is precipitated out. The crystals are filtered off, washed and recrystallised from 50 mL ethanol.

Yield: 7 g (68%; hydrochloride); m.p.=232-234° C.

### b) 8-{2-[1,1-dimethyl-2-(4-methoxy-phenyl)-ethylamino]-1-hydroxy-ethyl}-6-hydroxy-4H-benzo[1,4]oxazin-3-one

6.8 g of the benzyl compound obtained previously are hydrogenated in 125 mL methanol with the addition of 1 g palladium on charcoal (5%) at ambient temperature and normal pressure. The catalyst is filtered off and the filtrate is freed from solvent. After recrystallisation of the residue from 50 mL acetone and some water a solid is obtained which is filtered off and washed.

Yield: 5.0 g (89%; hydrochloride); m.p.=155-160° C.

The (R)- and (S)-enantiomers of Example 1 may be obtained from the racemate, for example, by means of chiral HPLC (e.g. column: Chirobiotic T, 250×22.1 mm supplied by Messrs Astec). The mobile phase used may be methanol with 0.05% triethylamine and 0.05% acetic acid. Silica gel with a particle size of 5 μm, to which the glycoprotein teicoplanin is covalently bound, may be used as column material.

Retention time (R-enantiomer)=40.1 min, retention time (S-enantiomer)=45.9 min. The two enantiomers are obtained by this method in the form of the free bases.

Of outstanding importance according to the invention is the R-enantiomer of Example 1.

### EXAMPLE 2

### 6-hydroxy-8-{1-hydroxy-2-[2-(ethyl 4-phenoxy-acetate)-1,1-dimethyl-ethylamino]-ethyl}-4H-benzo[1,4]oxazin-3-one

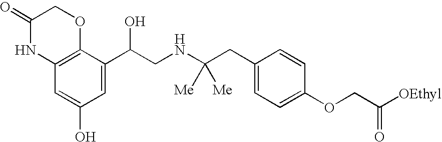

### a) 8-{2-[1,1-dimethyl-2-(ethyl 4-phenoxy-acetate)-ethylamino]-1-hydroxy-ethyl}-6-benzyloxy-4H-benzo[1,4]oxazin-3-one

Analogously to the method described in Example 1a) the title compound is obtained from 15 g (6-benzyloxy-4H-benzo[1,4]oxazin-3-one)-glyoxalhydrate and 11.8 g 1,1-dimethyl-2-(ethyl 4-phenoxy-acetate)-ethylamine hydrochloride.

Yield: 16.5 g (69%, hydrochloride); m.p.=212-214° C.

### b) 8-{2-[1,1-dimethyl-2-(4-phenoxy-acetate ethyl)-ethylamino]-1-hydroxy-ethyl]-6-hydroxy-4H-benzo[1,4]oxazin-3-one

8 g of the benzylalcohol obtained previously are dissolved in 100 mL ethanol, 100 mL methanol and 10 mL water and hydrogenated in the presence of 1 g palladium on charcoal (5%). After uptake of the theoretical amount of hydrogen calculated the catalyst is filtered off and the filtrate is evaporated down. The product that crystallises out when the solvent is distilled off is suction filtered and washed.

Yield: 5.5 g (81%; hydrochloride); m.p.=137-140° C.

The (R)- and (S)-enantiomers of this embodiment may be obtained by separation of the racemate analogously to common methods known in the art.

### EXAMPLE 3

### 6-hydroxy-8-{1-hydroxy-2-[2-(4-phenoxy-acetic acid)-1,1-dimethyl-ethylamino]-ethyl}4H-benzo[1,4]oxazin-3-one

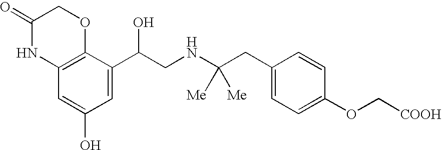

11 g 8-{2-[1,1-dimethyl-2-(4-phenoxy-acetate ethyl)-ethylamino]-1-hydroxy-ethyl}-6-benzyloxy-4H-benzo[1,4]oxazin-3-one hydrochloride (Example 4a) are dissolved in 125 mL methanol and hydrogenated in the presence of 1 g palladium on charcoal (5%). After uptake of the theoretically calculated amount of hydrogen the catalyst is filtered off. 2.6 g sodium hydroxide dissolved in 20 mL water are added to the filtrate. The mixture is refluxed for 30 minutes, the methanol is distilled off and the residue is combined with 10 mL water, 20 mL n-butanol and 3.9 mL acetic acid. The precipitated solid is suction filtered and washed with diethyl ether.

Yield: 7 g (87%). The hydrochloride is obtained by recrystallisation from 0.5 molar hydrochloric acid. M.p.=152° C.

The (R)- and (S)-enantiomers of this embodiment may be obtained by separation of the racemate analogously to common methods known in the art.

### EXAMPLE 4

### 8-{2-[1,1-dimethyl-2-(2,4,6-trimethylphenyl)-ethylamino]-1-hydroxy-ethyl}-6-hydroxy-4H-benzo[1,4]oxazin-3-one

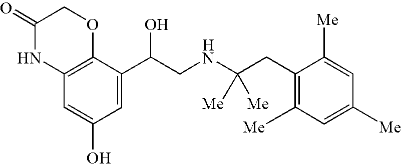

### a) 1-(6-benzyloxy-4H-benzo[1,4]oxazin-3-one)-2-[1,1-dimethyl-2-(2,4,6-trimethylphenyl)-ethylimino]-ethanone

7.2 g (6-benzyloxy-4H-benzo[1,4]oxazin-3-one)-glyoxalhydrate and 3.6 g 1,1-dimethyl-2-(2,4,6-trimethylphenyl)-ethylamine are heated to 70° C. for one hour in 100 mL ethanol. After cooling the solid precipitated is filtered off and washed with ethanol and diethyl ether. Yield: 8.6 g (94%); m.p.=175° C.

### b) 8-{2-[1,1-dimethyl-2-(2,4,6-trimethylphenyl)-ethylamino]-1-hydroxy-ethyl}-6-benzyloxy-4H-benzo[1,4]oxazin-3-one

8.6 g of the Schiff's base obtained according to the prescribed method 6a) are dissolved in 100 mL ethanol and 20 mL THF, combined within 30 min at 10-20° C. with 0.7 g sodium borohydride and stirred for one hour. After the addition of 10 mL acetone the mixture is stirred for 30 minutes and then diluted with ethyl acetate and water. The product that crystallises out during acidification with conc. hydrochloric acid is filtered off and washed.

Yield: 7.4 g (80%, hydrochloride); m.p.=235° C. (decomposition).

### c) 8-{2-[1,1-dimethyl-2-(2,4,6-trimethylphenyl)-ethylamino]-1-hydroxy-ethyl}-6-hydroxy-4H-benzo[1,4]oxazin-3-one

7.4 g of the benzyl compound obtained in Step b) are hydrogenated in 125 mL methanol with the addition of 1 g palladium on charcoal (5%) at ambient temperature and normal pressure. Then the catalyst is filtered off and the filtrate is evaporated down. The product that crystallises out on the addition of acetone is suction filtered and washed with acetone and diethyl ether. Yield: 5 g (78%, hydrochloride); m.p.=160° C. (decomposition).

The (R)- and (S)-enantiomers of this embodiment may be obtained by separation of the racemate analogously to common methods known in the art.

### EXAMPLE 5

### 6-hydroxy-8-{1-hydroxy-2-[2-(4-hydroxy-phenyl)-1,1-dimethyl-ethylamino]-ethyl}-4H-benzo[1,4]oxazin-3-one

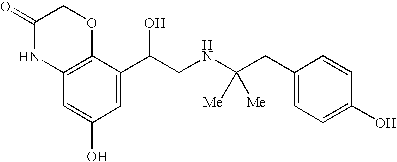

### a) 8-{2-[1,1-dimethyl-2-(4-hydroxy-phenyl)-ethylamino]-1-hydroxy-ethyl}-6-benzyloxy-4H-benzo[1,4]oxazin-3-one

The title compound is prepared from 10 g (6-benzyloxy-4H-benzo[1,4]oxazin-3-one)-glyoxalhydrate and 4.6 g 1,1-dimethyl-2-(4-hydroxy-phenyl)-ethylamine analogously to the procedure laid down for Example 1a).

Yield: 9.0 g (64%, hydrochloride); m.p.=255-258° C.

### b) 8-{2-[1,1-dimethyl-2-(4-hydroxy-phenyl)-ethylamino]-1-hydroxy-ethyl}-6-hydroxy-4H-benzo[1,4]oxazin-3-one

5.7 g of the coupling product obtained previously are hydrogenated in the presence of 0.6 g palladium on charcoal (5%) in 100 mL methanol. After uptake of the theoretically calculated amount of hydrogen the catalyst is filtered off and the filtrate is freed from solvent. The residue is dissolved in ethanol with heating and then combined with diethyl ether. The product precipitated is suction filtered and recrystallised once from water. Yield: 3.6 g (72%, hydrochloride); m.p.=159-162° C.

The (R)- and (S)-enantiomers of this embodiment may be obtained by separation of the racemate analogously to common methods known in the art.

### EXAMPLE 6

### 6-hydroxy-8-{1-hydroxy-2-[2-(4-isopropyl-phenyl)-1,1 dimethyl-ethylamino]-ethyl}-4H-benzo[1,4]oxazin-3-one

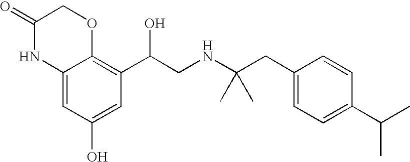

### a) 1-(4-isopropyl-phenyl)-2-methyl-propan-2-ol

The reaction of a Grignard compound, prepared from 20 g (119 mmol) 4-isopropylbenzyl chloride, with 11.4 ml (155 mmol) acetone yields the target compound as a colourless oil. Yield: 13.0 g (57%); mass spectrometry: [M+H]⁺=193.

### b) N-[2-(4-isopropyl-phenyl)-1,1-dimethyl-ethyl]-acetamide

A Ritter reaction is carried out with 10.2 g (53 mmol) 1-(4-isopropyl-phenyl)-2-methyl-propan-2-ol in the manner described for Example 7b). The reaction mixture is poured onto ice water and made alkaline with sodium hydroxide solution, during which time a solid is precipitated. This is suction filtered and dried.

Yield: 9.90 g (80%); mass spectrometry: [M+H]⁺=234.

### c) 2-(4-isopropyl-phenyl)-1,1-dimethyl-ethylamine

Reaction of 9.80 g (42 mmol) N-[2-(4-isopropyl-phenyl)-1,1-dimethyl-ethyl]-acetamide analogously to the procedure laid down for Example 7c).

Yield: 7.00 g (71%, hydrochloride); m.p.=202-206° C.

### d) 6-benzyloxy-8-{1-hydroxy-2-[2-(4-isopropyl-phenyl)-1,1-dimethyl-ethylamino]-ethyl}-4H-benzo[1,4]oxazin-3-one

2.18 g (6.1 mmol) benzyloxy-8-(2-ethoxy-2-hydroxy-acetyl)-4H-benzo[1,4]oxazin-3-one and 1.1 g (5.8 mmol) 2-(4-isopropyl-phenyl)-1,1-dimethyl-ethylamine are stirred for one hour at 50-80° C. in 40 mL ethanol. After cooling to ambient temperature 0.24 g (6.3 mmol) sodium borohydride are added. The mixture is stirred for one hour, diluted with 5 mL acetone and stirred for a further 30 minutes. The reaction mixture is acidified with hydrochloric acid, combined with 100 mL water and 80 mL ethyl acetate and made alkaline with ammonia. The organic phase is separated off, dried with sodium sulphate and freed from the solvent. The residue is dissolved in 20 mL ethyl acetate and 10 mL water, acidified with conc. hydrochloric acid and diluted with diethyl ether. After the addition of a crystallisation aid the precipitated solid is suction filtered and washed. White solid. Yield: 1.7 g (52%, hydrochloride); m.p.=220-222° C.

### e) 6-hydroxy-8-{1-hydroxy-2-[2-(4-isopropyl-phenyl)-1,1-dimethyl-ethylamino]-ethyl}-4H-benzo[1,4]oxazin-3-one

1.6 g (3.0 mmol) 6-benzyloxy-8-{1-hydroxy-2-[2-(4-isopropyl-phenyl)-1,1-dimethyl-ethylamino]-ethyl}-4H-benzo[1,4]oxazin-3-one are dissolved in methanol and hydrogenated with palladium on charcoal as catalyst at normal pressure and ambient temperature. The catalyst is suction filtered, the solvent distilled off and the residue recrystallised from isopropanol. White solid.

Yield: 1.1 g (85%, hydrochloride); m.p.=248-250° C.; mass spectrometry: [M+H]⁺=399.

The (R)- and (S)-enantiomers of this embodiment may be obtained by separation of the racemate analogously to common methods known in the art.

### EXAMPLE 7

### 8-{2-[2-(4-ethyl-phenyl)-1,1-dimethyl-ethylamino]-1-hydroxy-ethyl}-6-hydroxy-4H-benzo[1,4]oxazin-3-one

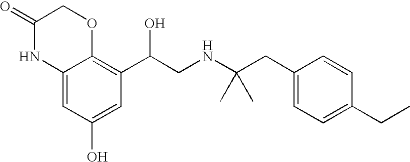

### a) 1-(4-ethyl-phenyl)-2-methyl-propan-2-ol

14.8 g (90 mmol) 1-(4-ethyl-phenyl)-propan-2-one, dissolved in diethyl ether, are added dropwise to 39 mL of a 3 molar solution of methylmagnesium bromide in diethyl ether, while being cooled with the ice bath, in such a way that the temperature does not exceed 30° C. After the addition has ended the reaction mixture is left to reflux for 1.5 hours and then hydrolysed with 10% ammonium chloride solution. After separation of the organic phase the aqueous phase is extracted with diethyl ether. The combined ether phases are washed with water, dried with sodium sulphate and evaporated down. The oil thus obtained is further reacted directly.

Yield: 15.5 g (90%).

### b) N-[2-(4-ethyl-phenyl)-1,1-dimethyl-ethyl]-acetamide

6.2 mL conc. sulphuric acid are added dropwise within 15 minutes to 15.5 g (87 mmol) 1-(4-ethyl-phenyl)-2-methyl-propan-2-ol in 4.8 mL (91 mmol) acetonitrile and 15 mL glacial acetic acid, while the temperature rises to 65° C. Then the mixture is stirred for one hour, diluted with ice water and made alkaline with conc. sodium hydroxide solution. After further stirring for 30 minutes the precipitated solid is suction filtered and washed with water. The crude product is dissolved in ethyl acetate, dried with sodium sulphate and evaporated down. The oil remaining is combined with petroleum ether, during which time a solid is precipitated, which is filtered off and dried.

Yield: 16.3 g (85%); m.p.=90-92° C.

### c) 2-(4-ethyl-phenyl)-1,1-dimethyl-ethylamine

16.3 g (74 mmol) N-[2-(4-ethyl-phenyl)-1,1-dimethyl-ethyl]-acetamide and 8.0 g potassium hydroxide are heated for 15 hours in 60 mL ethyleneglycol at reflux temperature. The reaction mixture is combined with ice water and extracted three times with diethyl ether. The combined organic phases are washed with water, dried with sodium sulphate and freed from the solvent. In order to prepare the hydrochloride the crude product is dissolved in acetonitrile and combined successively with ethereal hydrochloric acid and diethyl ether. The precipitated solid is suction filtered and dried.

Yield: 11.0 g (69%, hydrochloride); m.p.=165-167° C.

### d) 6-benzyloxy-8-{2-[2-(4-ethyl-phenyl)-1,1-dimethyl-ethylamino]-1-hydroxy-ethyl}-4H-benzo[1,4]oxazin-3-one

The target compound is prepared analogously to the procedure laid down for Example 6d) from 2.14 g (6.0 mmol) 6-benzyloxy-8-(2-ethoxy-2-hydroxy-acetyl)-4H-benzo[1,4]oxazin-3-one and 1.0 g (5.6 mmol) 2-(4-ethyl-phenyl)-1,1-dimethyl-ethylamine. White solid. Yield: 1.7 g (54%, hydrochloride); m.p. 210-214° C.

### e) 8-{2-[2-(4-ethyl-phenyl)-1,1-dimethyl-ethylamino]-1-hydroxy-ethyl}-6-hydroxy-4H-benzo[1,4]oxazin-3-one

The hydrogenolysis of 1.45 g (2.75 mmol) 6-benzyloxy-8-{2-[2-(4-ethyl-phenyl)-1,1-dimethyl-ethylamino]-1-hydroxy-ethyl}-4H-benzo[1,4]oxazin-3-one according to the prescribed method for Example 6e) yields the target compound in the form of a white solid.

Yield: 1.07 g (92%; hydrochloride); m.p.=266-269° C.; mass spectrometry: [M+H]⁺=385.

The (R)- and (S)-enantiomers of this embodiment may be obtained by separation of the racemate analogously to common methods known in the art.

### EXAMPLE 8

### 8-{2-[2-(4-Fluoro-3-methyl-phenyl)-1,1-dimethyl-ethylamino]-1-hydroxy-ethyl}-6-hydroxy-4H-benzo[1,4]oxazin-3-one

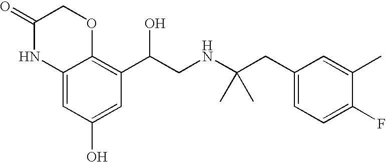

### a) 1-fluoro-2-methyl-4-(2-methyl-propenyl)-benzene

100 mL of a 0.5 molar solution of 4-fluoro-3-methyl-phenylmagnesium bromide in THF are combined within 30 minutes with 4.7 mL (50 mmol) isopropylaldehyde, while the temperature rises to 45° C. It is stirred for 30 minutes, refluxed for 1 hour and then hydrolysed with 10% ammonium chloride solution. After separation of the organic phase the mixture is extracted with diethyl ether. The organic phases are combined, dried and evaporated down. The alcohol thus obtained is dissolved in 100 mL toluene, combined with 1 g of p-toluenesulphonic acid monohydrate and refluxed for three hours using the water separator. The reaction mixture is poured onto water and made alkaline with conc. sodium hydroxide solution. After separation of the organic phase this is washed with water, dried with sodium sulphate and freed from the solvent. Fractionated distillation of the residue yields the product in the form of a colourless liquid (b.p. 80-85° C./10 mbar). Yield: 4.1 g (50%).

### b) N-[2-(4-fluoro-3-methyl-phenyl)-1,1-dimethyl-ethyl]-formamide

4.9 mL conc. sulphuric acid are added dropwise at 5-15° C. to 1.5 g (31 mmol) sodium cyanide in 5 mL glacial acetic acid. Then the mixture is combined with 3.9 g (24 mmol) 1-fluoro-2-methyl-4-(2-methyl-propenyl)-benzene, dissolved in 10 mL glacial acetic acid, and stirred for 1 hour at 50-60° C. The reaction mixture is diluted with ice water, made alkaline with conc. sodium hydroxide solution and extracted with dichloromethane. The organic phase is dried with sodium sulphate and freed from the solvent in vacuo. The light yellow oil thus obtained is further reacted directly. Yield: 4.3 g (87%).

### c) 2-(4-fluoro-3-methyl-phenyl)-1,1-dimethyl-ethylamine

4.3 g (20.6 mmol) N-[2-(4-fluoro-3-methyl-phenyl)-1,1-dimethyl-ethyl]-formamide, 20 mL conc. hydrochloric acid and 20 mL water are refluxed for 2 hours. The reaction mixture is diluted with water, made alkaline with conc. sodium hydroxide solution and extracted with dichloromethane. The organic phases are dried with sodium sulphate and evaporated down. The residue is dissolved in ethyl acetate, combined with ethereal hydrochloric acid and cooled. The precipitated crystals are suction filtered and washed with diethyl ether and dried. White solid.

Yield: 3.9 g (87%, hydrochloride); m.p.=196-198° C.

### d) 6-benzyloxy-8-{2-[2-(4-fluoro-3-methyl-phenyl)-1,1-dimethyl-ethylamino]-1-hydroxy-ethyl}-4H-benzo[1,4]oxazin-3-one

1.10 g (3.1 mmol) benzyloxy-8-(2-ethoxy-2-hydroxy-acetyl)-4H-benzo[1,4]oxazin-3-one and 0.50 g (2.8 mmol) 2-(4-fluoro-3-methyl-phenyl)-1,1-dimethyl-ethylamine are reacted and worked up analogously to the procedure laid down for Example 6d). White solid.

Yield: 0.75 g (47%, hydrochloride); m.p.=228-230° C.

### e) 8-{2-[2-(4-Fluoro-3-methyl-phenyl)-1,1-dimethyl-ethylamino]-1-hydroxy-ethyl}-6-hydroxy-4H-benzo[1,4]oxazin-3-one

The hydrogenation of 0.70 g (1.4 mmol) 6-benzyloxy-8-{2-[2-(4-fluoro-3-methyl-phenyl)-1,1-dimethyl-ethylamino]-1-hydroxy-ethyl}-4H-benzo[1,4]oxazin-3-one yields the target compound as a white solid.

Yield: 0.50 g (87%, hydrochloride); m.p.=278-280° C.; mass spectroscopy: [M+H]⁺=389.

The (R)- and (S)-enantiomers of this embodiment may be obtained by separation of the racemate analogously to common methods known in the art.

### EXAMPLE 9

### 8-{2-[2-(4-fluoro-2-methyl-phenyl)-1,1-dimethyl-ethylamino]-1-hydroxy-ethyl}-6-hydroxy-4H-benzo[1,4]oxazin-3-one

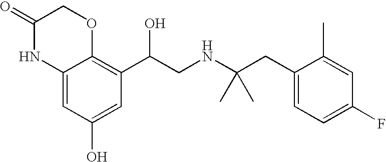

### a) 1-(4-fluoro-2-methyl-phenyl)-2-methyl-propyl acetate

500 mL of a 0.5 molar solution of 4-fluoro-6-methylphenylmagnesium bromide and 23.2 mL (260 mmol) isopropylaldehyde are reacted analogously to Example 8a). After hydrolysis with 10% ammonium chloride solution the aqueous phase is separated off and extracted with diethyl ether. The combined organic phases are dried with sodium sulphate and evaporated down. The alcohol thus obtained is then dissolved in 50 mL acetic anhydride, combined with 1 mL conc. sulphuric acid and stirred for three hours at reflux temperature. Then the reaction mixture is poured onto water, stirred for a further hour and made alkaline. The mixture is extracted with dichloromethane, the organic phases are dried with sodium sulphate and the solvents are distilled off. Fractional distillation of the residue yields the product in the form of a colourless liquid (b.p. 105-110° C./8 mbar). Yield 29.0 g (52%).

### b) N-[2-(4-fluoro-2-methyl-phenyl)-1,1-dimethyl-ethyl]-formamide

29.0 g (130 mmol) 1-(4-fluoro-2-methyl-phenyl)-2-methyl-propyl acetate are reacted and worked up analogously to the procedure laid down for Example 8b). Yellow oil. Yield: 27.0 g (99%).

### c) 2-(4-fluoro-2-methyl-phenyl)-1,1-dimethyl-ethylamine

In order to prepare the amine 27.0 g (130 mmol) N-[2-(4-fluoro-2-methyl-phenyl)-1,1-dimethyl-ethyl]-formamide are reacted as in the procedure laid down for Example 8c). White solid. Yield: 15.5 g (55%, hydrochloride); m.p.=277-280° C.

### d) 6-benzyloxy-8-{2-[2-[4-fluoro-2-methyl-phenyl)-1,1-dimethyl-ethylamino]-1-hydroxy-ethyl}-4H-benzo[1,4]oxazin-3-one

Prepared analogously to the procedure laid down for Example 6d) from 0.95 g (2.66 mmol) benzyloxy-8-(2-ethoxy-2-hydroxy-acetyl)-4H-benzo[1,4]oxazin-3-one and 0.43 g (2.37 mmol) 2-(4-fluoro-2-methyl-phenyl)-1,1-dimethyl-ethylamine.

Yield: 0.75 g (55%, hydrochloride); m.p.=233-236° C.

### e) 8-{2-[2-(4-fluoro-2-methyl-phenyl)-1,1-dimethyl-ethylamino]-1-hydroxy-ethyl}-6-hydroxy-4H-benzo[1,4]oxazin-3-one

The debenzylation of 0.70 g (1.36 mmol) 6-benzyloxy-8-{2-[2-[4-fluoro-2-methyl-phenyl)-1,1-dimethyl-ethylamino]-1-hydroxy-ethyl}-4H-benzo[1,4]oxazin-3-one yields the target compound in the form of a white solid.

Yield: 0.50 g (87%, hydrochloride); m.p.=278-280° C.; mass spectroscopy: [M+H]⁺=389.

The (R)- and (S)-enantiomers of this embodiment may be obtained by separation of the racemate analogously to common methods known in the art.

### EXAMPLE 10

### 8-{2-[2-(2,4-difluoro-phenyl)-1,1-dimethyl-ethylamino]-1-hydroxy-ethyl}-6-hydroxy-4H-benzo[1,4]oxazin-3-one

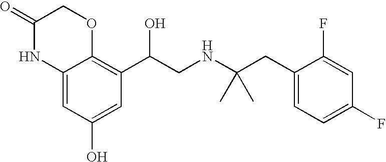

### a) 1-(2,4-difluoro-phenyl)-2-methyl-propan-2-ol

11.0 mL acetone, diluted with 50 mL diethyl ether, are added dropwise within 20 minutes to a solution of 500 mL 0.25 molar 2,4-difluorobenzylmagnesium bromide in diethyl ether. Then the mixture is stirred for 1.5 hours at reflux temperature and then hydrolysed with 10% ammonium chloride solution. The ether phase is separated off, washed with water, dried with sodium sulphate and evaporated down. The fractional distillation of the residue yields the alcohol as a colourless liquid (b.p. 70-73° C./2 mmbar).

Yield: 20.0 g (86%).

### b) N-[2-(2,4-difluoro-phenyl]-1,1-dimethyl-ethyl]-formamide

Ritter reaction with 20 g (110 mmol) 1-(2,4-difluoro-phenyl)-2-methyl-propan-2-ol according to the process described for Example 8b). Yellow oil. Yield: 22.0 g (94%).

### c) 2-(2,4-difluoro-phenyl)-1,1-dimethyl-ethylamine

Reaction of 22.0 g (100 mmol) N-[2-(2,4-difluoro-phenyl]-1,1-dimethyl-ethyl]-formamide analogously to the procedure laid down for Example 8c).

Yield: 16.0 g (72%, hydrochloride); m.p.=201-203° C.

### d) 6-benzyloxy-8-{2-[2-(2,4-difluoro-phenyl)-1,1-dimethyl-ethylamino]-1-hydroxy-ethl}-4H-benzo[1,4]oxazin-3-one

Reaction of 0.89 g (2.49 mmol) benzyloxy-8-(2-ethoxy-2-hydroxy-acetyl)-4H-benzo[1,4]oxazin-3-one and 0.40 g (2.16 mmol) 2-(2,4-difluoro-phenyl)-1,1-dimethyl-ethylamine in the manner described for Example 6d).

Yield: 0.80 g (62%, hydrochloride); m.p.=245-247° C.

### e) 8-{2-[2-(2,4-difluoro-phenyl)-1,1-dimethyl-ethylamino]-1-hydroxy-ethyl}-6-hydroxy-4H-benzo[1,4]oxazin-3-one

The hydrogenolysis of 0.70 g (1.35 mmol) 6-benzyloxy-8-{2-[2-(2,4-difluoro-phenyl)-1,1-dimethyl-ethylamino]-1-hydroxy-ethyl}-4H-benzo[1,4]oxazin-3-one yields the target compound as a white solid.

Yield: 0.48 g (83%, hydrochloride); m.p.=279-280° C.; mass spectroscopy: [M+H]⁺=393.

The (R)- and (S)-enantiomers of this embodiment may be obtained by separation of the racemate analogously to common methods known in the art.

### EXAMPLE 11

### 8-{2-[2-(3,5-difluoro-phenyl)-1,1-dimethyl-ethylamino]-1-hydroxy-ethyl}-6-hydroxy-4H-benzo[1,4]oxazin-3-one

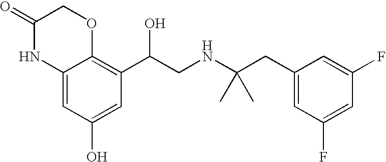

### a) 1-(3,5-difluoro-phenyl)-2-methyl-propan-2-ol

The target compound is obtained by reacting a Grignard compound, prepared from 25.0 g (121 mmol) 3,5-difluorobenzylbromide, with 12.6 mL (171 mmol) acetone. Yellow oil.

Yield: 13.5 g (60%).

### b) 2-(3,5-difluoro-phenyl)-1,1-dimethyl-ethylamine

The Ritter reaction of 5.5 g (29.5 mmol) 1-(3,5-difluoro-phenyl)-2-methyl-propan-2-ol and 1.8 g sodium cyanide yields 7.0 g formamide, which is treated with hydrochloric acid in order to cleave the formyl group. Light yellow oil. Yield: 4.6 g (75%).

### c) 6-benzyloxy-8-{2-[2-(3,5-difluoro-phenyl)-1,1-dimethyl-ethylamino]-1-hydroxy-ethyl}-4H-benzo[1,4]oxazin-3-one

Prepared from 1.73 g (4.84 mmol) benzyloxy-8-(2-ethoxy-2-hydroxy-acetyl)-4H-benzo[1,4]oxazin-3-one and 0.80 g (4.32 mmol) 2-(3,5-difluoro-phenyl)-1,1-dimethyl-ethylamine in the usual way.

Yield: 1.50 g (58%, hydrochloride); m.p.=240-244° C.

### d) 8-[2-[2-(3,5-difluoro-phenyl)-1,1-dimethyl-ethylamino]-1-hydroxy-ethyl}-6-hydroxy-4H-benzo[1,4]oxazin-3-one

Hydrogenolysis of 1.30 g (2.43 mmol) 6-benzyloxy-8-{2-[2-(3,5-difluoro-phenyl)-1,1-dimethyl-ethylamino]-1-hydroxy-ethyl}-4H-benzo[1,4]oxazin-3-one yields the target compound as a white solid.

Yield: 0.90 g (86%, hydrochloride); m.p.=150-158° C.; mass spectroscopy: [M+H]⁺=393.

The (R)- and (S)-enantiomers of this embodiment may be obtained by separation of the racemate analogously to common methods known in the art.

### EXAMPLE 12

### 8-{2-[2-(4-ethoxy-phenyl)-1,1-dimethyl-ethylamino]-1-hydroxy-ethyl}-6-hydroxy-4H-benzo[1,4]oxazin-3-one

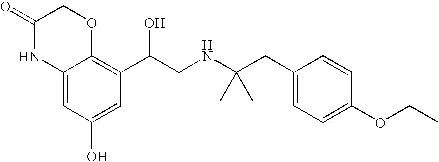

### a) benzyl [2-(4-ethoxy-phenyl)-1,1-dimethyl-ethyl]-carbamate

15.0 g (50 mmol) benzyl [2-(4-hydroxy-phenyl)-1,1-dimethyl-ethyl]-carbamate are stirred with 7.5 mL (92 mmol) ethyl iodide and 21 g (150 mmol) potassium carbonate for 10 hours at 90-100° C. The reaction mixture is combined with ethyl acetate, washed twice with water and dried with sodium sulphate. After removal of the solvents by distillation a yellow oil remains (15.0 g, 92%), which is further reacted directly.

### b) 2-(4-ethoxy-phenyl)-1,1-dimethyl-ethylamine

A solution of 15.0 g (49 mmol) benzyl [2-(4-ethoxy-phenyl)-1,1-dimethyl-ethyl]-carbamate in 100 mL glacial acetic acid is combined with 2 g palladium on charcoal (10%) and then hydrogenated at 5 bar and 40 to 50° C. The catalyst is filtered off and the filtrate is freed from solvent. The residue is dissolved in a little water, made alkaline with conc. sodium hydroxide solution and extracted with ethyl acetate. The organic phase is washed with water, dried with sodium sulphate and evaporated down. The crude product is dissolved in acetonitrile and acidified with ethereal hydrochloric acid. The solid precipitated after the addition of diethyl ether is suction filtered and dried.

Yield: 8.8 g (hydrochloride, 84%); m.p.=198-200° C.

### c) 6-benzyloxy-8-{2-[2-(4-ethoxy-phenyl)-1,1-dimethyl-ethylamino]-1-hydroxy-ethyl}-4H-benzo[1,4]oxazin-3-one

2.14 g (6.0 mmol) 6-benzyloxy-8-(2-ethoxy-2-hydroxy-acetyl)-4H-benzo[1,4]oxazin-3-one and 1.0 g (5.2 mmol) 2-(4-ethoxy-phenyl)-1,1-dimethyl-ethylamine are stirred in 40 mL ethanol for one hour at 50-80° C. After cooling to ambient temperature 0.23 g (6.0 mmol) sodium borohydride are added and the mixture is stirred for another hour. The reaction mixture is combined with 5 ml acetone, stirred for 30 minutes, acidified with glacial acetic acid and evaporated down. The residue is combined with water and ethyl acetate and made alkaline. The organic phase is separated off, washed with water, dried with sodium sulphate and freed from the solvent in vacuo. The residue is dissolved again in ethyl acetate and water, combined with conc. hydrochloric acid and diluted with diethyl ether. The precipitated solid is suction filtered and washed with diethyl ether. White solid.

Yield: 2.0 g (61%, hydrochloride); m.p.=214-216° C.

### d) 8-{2-[2-(4-ethoxy-phenyl)-1,1-dimethyl-ethylamino]-1-hydroxy-ethyl}-6-hydroxy-4H-benzo[1,4]oxazin-3-one

1.5 g (2.8 mmol) 6-benzyloxy-8-{2-[2-(4-ethoxy-phenyl)-1,1-dimethyl-ethylamino]-1-hydroxy-ethyl}-4H-benzo[1,4]oxazin-3-one in 80 mL methanol are hydrogenated with 250 mg palladium on charcoal (10%) as catalyst at ambient temperature and normal pressure. The catalyst is suction filtered and the filtrate is evaporated down. The residue is dissolved in 5 mL ethanol by heating, inoculated and diluted with ethyl acetate. The precipitated solid is filtered off and washed. White solid.

Yield 1.0 g (83%, hydrochloride); m.p=232-235° C.; mass spectrometry: [M+H]⁺=401.

The (R)- and (S)-enantiomers of this embodiment may be obtained by separation of the racemate analogously to common methods known in the art.

### EXAMPLE 13

### 8-{2-[2-(3,5-dimethyl-phenyl)-1,1-dimethyl-ethylamino]-1-hydroxy-ethyl}-6-hydroxy-4H-benzo[1,4]oxazin-3-one

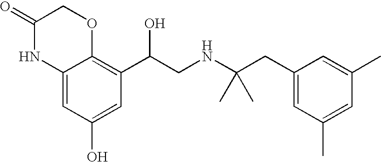

### a) 1-(3,5-dimethyl-phenyl)-2-methyl-propanol-2-ol

Obtained from the reaction of ethyl (3,5-dimethyl-phenyl)-acetate with methylmagnesium bromide.

### b) 2-(3,5-dimethyl-phenyl)-1,1-dimethyl-ethylamine

By reacting 6.00 g (34 mmol) 1-(3,5-dimethyl-phenyl)-2-methyl-propanol-2-ol and 2.00 g (41 mmol) sodium cyanide in a Ritter reaction 2.40 g 2-(3,5-dimethyl-phenyl)-1,1-dimethyl-ethylformamid (35% yield) are obtained. To release the the amine the formamide (2.40 g, 11.7 mmol) is treated with hydrochloric acid. The process and working up take place analogously to the procedure laid down for Example 8c). Oil. Yield: 1.70 g (82%); mass spectroscopy: [M+H]⁺=178.

### c) 6-benzyloxy-8-{2-[2-(3,5-dimethyl-phenyl)-1,1-dimethyl-ethylamino]-1-hydroxy-ethyl}-4H-benzo[1,4]oxazin-3-one

Prepared analogously to the procedure laid down for Example 8d) from 1.47 g (4.1 mmol) benzyloxy-8-(2-ethoxy-2-hydroxy-acetyl)-4H-benzo[1,4]oxazin-3-one and 0.65 g (3.7 mmol) 2-(3,5-dimethyl-phenyl)-1,1-dimethyl-ethylamine.

Yield: 1.1 g (51%, hydrochloride); m.p.=220-222° C.

### d) 8-{2-[2-(3,5-dimethyl-phenyl)-1,1-dimethyl-ethylamino]-1-hydroxy-ethyl}-6-hydroxy-4H-benzo[1,4]oxazin-3-one

The target compound was obtained after hydrogenolysis of 0.90 g (1.71 mmol) 6-benzyloxy-8-{2-[2-(3,5-dimethyl-phenyl)-1,1-dimethyl-ethylamino]-1-hydroxy-ethyl}-4H-benzo[1,4]oxazin-3-one and recrystallisation of the crude product from isopropanol. White solid.

Yield: 0.50 g (69%, hydrochloride); m.p.=235-238° C.; mass spectroscopy: [M+H]⁺=385.

The (R)- and (S)-enantiomers of this embodiment may be obtained by separation of the racemate analogously to common methods known in the art.

### EXAMPLE 14

### 4-(4-{2-[2-hydroxy-2-(6-hydroxy-3-oxo-3,4-dihydro-2H-benzo[1,4]oxazin-8-yl)-ethylamino]-2-methyl-propyl}-phenoxy)-butyric acid

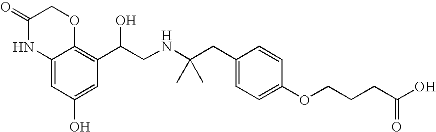

### a) ethyl 4-[4-(2-amino-2-methyl-propyl)-phenoxy]-butyrate

4.5 g (15.0 mmol) benzyl [2-(4-hydroxy-phenyl)-1,1-dimethyl-ethyl]-carbamate, 2.3 mL (16.0 mmol) ethyl 4-bromo-butyrate, 2.3 g (16.6 mmol) potassium carbonate and 0.3 g (1.8 mmol) potassium iodide in 20 mL dimethylformamid are heated to 120° C. for 13 hours. The reaction mixture is diluted with ethyl acetate and washed successively with water, sodium hydroxide solution and water. The organic phase is dried with sodium sulphate and evaporated down. The residue is purified by chromatography (eluant: cyclohexane/ethyl acetate=9:1). 5.0 g of a yellow oil are isolated, which is dissolved in 50 mL acetic acid and hydrogenated with 1.0 g palladium on charcoal as catalyst at 40° C. and 3 bar. The catalyst is filtered off and the filtrate is freed from solvent. The residue is dissolved in diethyl ether and combined with ethereal hydrochloric acid. The precipitated solid is suction filtered and dried.

Yield: 2.9 g (66% over two steps, hydrochloride); m.p.=103-105° C.

### b) 4-(4-{2-[2-(6-benzyloxy-3-oxo-3,4-dihydro-2H-benzo[1,4]oxazin-8-yl)-2-hydroxy-ethylamino]-2-methyl-propyl}-phenoxy)-butyrate ethyl

1.20 g (3.36 mmol) benzyloxy-8-(2-ethoxy-2-hydroxy-acetyl)-4H-benzo[1,4]oxazin-3-one and 0.90 g (3.22 mmol) ethyl 4-[4-(2-amino-2-methyl-propyl)-phenoxy]-butyrate are reacted in the manner described for Example 8d). The crude product is dissolved in 10 mL ethyl acetate and 10 mL water and combined with oxalic acid with stirring. The solution is diluted with diethyl ether and the precipitated solid is suction filtered and washed with diethyl ether.

Yield: 1.20 g (54%, oxalate); m.p.=223-227° C.

### c) 4-(4-{2-[2-(6-benzyloxy-3-oxo-3,4-dihydro-2H-benzo[1,4]oxazin-8-yl)-2-hydroxy-ethylamino]-2-methyl-propyl}-phenoxy)-butyric acid

A solution of 1.00 g (1.73 mmol) ethyl 4-(4-{2-[2-(6-benzyloxy-3-oxo-3,4-dihydro-2H-benzo[1,4]oxazin-8-yl)-2-hydroxy-ethylamino]-2-methyl-propyl}-phenoxy)-butyrate in 25 mL methanol is combined with 2.5 mL 1 N sodium hydroxide solution, refluxed for 30 minutes and then neutralised with 1 N hydrochloric acid. The solution is evaporated down and the oil remaining is dissolved in 5 mL n-butanol by heating. After the addition of a crystallisation aid a solid is precipitated, which is suction filtered and washed with acetone and diethyl ether. Yield: 0.75 g (79%); m.p.=216-218° C.

### d) 4-(4-{2-[2-hydroxy-2-(6-hydroxy-3-oxo-3,4-dihydro-2H-benzo[1,4]oxazin-8-yl)-ethylamino]-2-methyl-propyl}-phenoxy)-butyric acid

0.70 g (1.28 mmol) 4-(4-{2-[2-(6-benzyloxy-3-oxo-3,4-dihydro-2H-benzo[1,4]oxazin-8-yl)-2-hydroxy-ethylamino]-2-methyl-propyl}-phenoxy)-butyric acid are dissolved in 25 mL methanol and 2 mL acetic acid and hydrogenated in the presence of 150 mg palladium on charcoal (10%) at ambient temperature and normal pressure. The catalyst is filtered off and the filtrate is freed from solvent. The product is obtained by crystallisation from a methanol/acetone mixture.

Yield: 0.40 g (68%); m.p.=201-204° C.; mass spectroscopy: [M+H]⁺=459.

The (R)- and (S)-enantiomers of this embodiment may be obtained by separation of the racemate analogously to common methods known in the art.

### EXAMPLE 15

### 8-{2-[2-(3,4-difluoro-phenyl)-1,1-dimethyl-ethylamino]-1-hydroxy-ethyl}-6-hydroxy-4H-benzo[1,4]oxazin-3-one

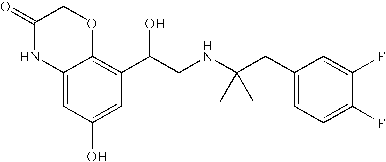

### a) 1-(3,4-difluoro-phenyl)-2-methyl-propan-2-ol

From 23.0 g (111 mmol) 3,4-difluorobenzylbromide a Grignard compound is prepared, which is then reacted with 11.6 mL (158 mmol) acetone. Light yellow oil.

Yield: 9.7 g (47%); R_{f} value: 0.55 (ethyl acetate/petroleum ether=1:3).

### b) N-[2-(3,4-difluoro-phenyl)-1,1-dimethyl-ethyl]-formamide

The target compound is obtained by a Ritter reaction with 4.0 g (21.5 mmol) 1-(3,4-difluoro-phenyl)-2-methyl-propan-2-ol. Light yellow oil.

Yield: 4.0 g (87%); mass spectrometry: [M+H]⁺=214.

### c) 2-(3,4-difluoro-phenyl)-1,1-dimethyl-ethylamine

4.00 g (18.5 mmol) N-[2-(3,4-difluoro-phenyl)-1,1-dimethyl-ethyl]-formamide are dissolved in ethanol, combined with conc. hydrochloric acid and heated overnight at reflux temperature. The reaction solution is poured onto ice water, made alkaline with sodium hydroxide and extracted with tert-butylmethylether. The organic phases are washed with water, dried with sodium sulphate and evaporated down. Yellow oil.

Yield: 3.2 g (92%); mass spectrometry: [M+H]⁺=186.

### d) 8-{2-[2-(3,4-difluoro-phenyl)-1,1-dimethyl-ethylamino]-1-hydroxy-ethyl}-6-hydroxy-4H-benzo[1,4]oxazin-3-one

357 mg (1 mmol) 6-benzyloxy-8-(2-ethoxy-2-hydroxy-acetyl)-4H-benzo[1,4]oxazin-3-one and 185 mg (1 mmol) 2-(3,4-difluoro-phenyl)-1,1-dimethyl-ethylamine are stirred for 30 minutes in 5 mL tetrahydrofuran at ambient temperature. The mixture is cooled to 0° C. and under an argon atmosphere 1.5 mL of a 2 molar solution of lithium borohydride in tetrahydrofuran is added dropwise. The mixture is stirred for 30 min at ambient temperature, combined with 10 mL dichloromethane and 3 mL water, stirred for a further hour and then filtered through Extrelut®. The eluate containing the ethanolamine is freed from the solvent. The residue is dissolved in methanol and hydrogenated with palladium on charcoal (10%) as catalyst at 2.5 bar and ambient temperature. Then the catalyst is separated off and the crude product purified by chromatography. White solid.

Yield: 31 mg (6%, trifluorethyl acetate); mass spectroscopy: [M+H]⁺=393.

The (R)- and (S)-enantiomers of this embodiment may be obtained by separation of the racemate analogously to common methods known in the art.

### EXAMPLE 16

### 8-{2-[2-(2-chloro-4-fluoro-phenyl)-1,1-dimethyl-ethylamino]-1-hydroxy-ethyl}-6-hydroxy-4H-benzo[1,4]oxazin-3-one

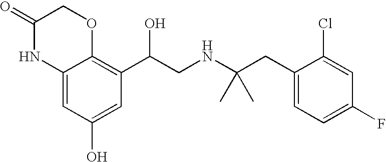

### a) 1-(2-chloro-4-fluoro-phenyl)-2-methyl-propan-2-ol

Prepared from 20 g (97 mmol) methyl (2-chloro-4-fluoro-phenyl)-acetate and 98 mL of a 3 molar solution of methylmagnesium bromide analogously to the procedure laid down for Example 6a).

### b) N-[2-(2-chloro-4-fluoro-phenyl)-1,1-dimethyl-ethyl]-formamide

7.5 g (37 mmol) 1-(2-chloro-4-fluoro-phenyl)-2-methyl-propan-2-ol were reacted and worked up according to the procedure described for Example 8b). The oil thus obtained was chromatographed for further purification on a short silica gel column (petroleum ether/ethyl acetate=9:1). Oil. Yield 7.4 g (87%); mass spectrometry: [M+H]⁺=230/232.

### c) 2-(2-chloro-4-fluoro-phenyl)-1,1-dimethyl-ethylamine

Reaction of 7.4 g (32 mmol) N-[2-(2-chloro-4-fluoro-phenyl)-1,1-dimethyl-ethyl]-formamide as in the procedure laid down for Example 15c) described. Brown oil. Yield: 5.14 g (79%); mass spectrometry: [M+H]⁺=202/204.

### d) 8-{2-[2-(2-chloro-4-fluoro-phenyl)-1,1-dimethyl-ethylamino]-1-hydroxy-ethyl}-6-hydroxy-4H-benzo[1,4]oxazin-3-one

357 mg (1 mmol) 6-benzyloxy-8-(2-ethoxy-2-hydroxy-acetyl)-4H-benzo[1,4]oxazin-3-one and 202 mg (1 mmol) 2-(2-chloro-4-fluoro-phenyl)-1,1-dimethyl-ethylamine are reacted with lithium borohydride analogously to the procedure laid down for Example 8d). For debenzylation of the ethanolamine thus obtained the latter is dissolved in 3 mL dichloromethane and cooled to −78° C. At this temperature 2 ml of a 1 molar solution of boron tribromide in dichloromethane is added dropwise and the mixture is allowed to come slowly up to ambient temperature. The reaction mixture is combined with 10 mL dichloromethane and 3 mL water and filtered through Extrelut®. The eluate is freed from the solvent and the residue is purified by chromatography. White solid. Yield: 70 mg (13%, trifluorethyl acetate); mass spectroscopy: [M+H]⁺=409/11.

The (R)- and (S)-enantiomers of this embodiment may be obtained by separation of the racemate analogously to common methods known in the art.

### EXAMPLE 17

### 8-{2-[2-(4-chloro-phenyl)-1,1-dimethyl-ethylamino]-1-hydroxy-ethyl}-6-hydroxy-4H-benzo[1,4]oxazin-3-one

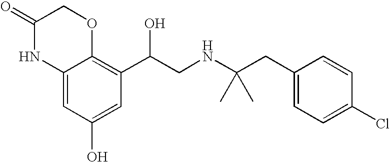

A solution of 300 mg (0.91 mmol) 6-benzyloxy-8-(2,2-dihydroxy-acetyl)-4H-benzo[1,4]oxazin-3-one and 200 mg (1.09 mmol) 2-(4-chloro-phenyl)-1,1-dimethyl-ethylamine in 3 mL ethanol was combined with molecular sieve and stirred for 90 minutes at 80° C. The mixture was left to cool to ambient temperature, 35 mg (0.91 mmol) sodium borohydride were added and the mixture was stirred for 1 hour. Then the reaction mixture was combined with sodium hydrogen carbonate solution and extracted with ethyl acetate. The combined organic phases were freed from the solvent and the residue was chromatographed (eluant: hexane/ethyl acetate/methanol), producing 305 mg ethanolamine. This was dissolved in 3 mL dichloromethane and cooled to −78° C. under an argon atmosphere. 3 mL of a 1 molar solution of boron tribromide in dichloromethane were added dropwise and the mixture was left for one hour at −78° C. and 20 minutes at ambient temperature with stirring. Then at −78° C. 3 mL conc. ammonia solution was added dropwise and the mixture was stirred for 5 minutes. The reaction mixture was combined with ammonium chloride solution and extracted with ethyl acetate. The combined organic phases were evaporated down and the residue was chromatographed for further purification (silica gel; eluant: dichloromethane/methanol+1% ammonia). Beige solid: 93 mg (26%); mass spectrometry: [M+H]⁺=391.

The (R)- and (S)-enantiomers of this embodiment may be obtained by separation of the racemate analogously to common methods known in the art.

### EXAMPLE 18

### 8-{2-[2-(4-bromo-phenyl)-1,1-dimethyl-ethylamino]-1-hydroxy-ethyl}-6-hydroxy-4H-benzo[1,4]oxazin-3-one

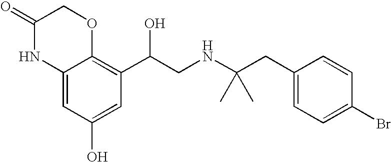

The preparation of the ethanolamine and debenzylation were carried out as described for Example 17 from 300 mg (0.91 mmol) 6-benzyloxy-8-(2,2-dihydroxy-acetyl)-4H-benzo[1,4]oxazin-3-one and 250 mg (1.09) mmol) 2-(4-bromo-phenyl)-1,1-dimethyl-ethylamine. Beige solid. Yield: 54 mg (14%); mass spectrometry: [M+H]⁺=435, 437.

The (R)- and (S)-enantiomers of this embodiment may be obtained by separation of the racemate analogously to common methods known in the art.

### EXAMPLE 19

### 8-{2-[2-(4-fluoro-phenyl)-1,1-dimethyl-ethylamino]-1-hydroxy-ethyl}-6-hydroxy-4H-benzo[1,4]oxazin-3-one

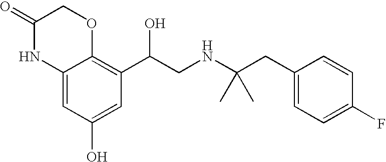

300 mg (0.91 mmol) 6-benzyloxy-8-(2,2-dihydroxy-acetyl)-4H-benzo[1,4]oxazin-3-one and 183 mg (1.09 mmol) 2-(4-fluoro-phenyl)-1,1-dimethyl-ethylamine were dissolved in 3 ml ethanol. Molecular sieve was added and the mixture was heated to 80° C. for 30 minutes. After cooling to ambient temperature, 35 mg (0.91 mmol) sodium borohydride were added. The mixture was stirred for 1 hour at ambient temperature, then sodium hydrogen carbonate solution was added to the reaction mixture and it was extracted with ethyl acetate. The organic phases were evaporated down and the residue was chromatographed (eluant: hexane/ethyl acetate/methanol). The ethanolamine thus obtained (223 mg) was dissolved in methanol in order to cleave the benzyl protecting group and hydrogenated with 150 mg palladium hydroxide as catalyst at ambient temperature and normal pressure. The catalyst was separated off by filtration through Celite®, the filtrate was freed from solvent and the residue was chromatographed (silica gel; eluant: dichloromethane/methanol). Beige solid.

Yield: 76 mg (22%); mass spectrometry: [M+H]⁺=375.

The (R)- and (S)-enantiomers of this embodiment may be obtained by separation of the racemate analogously to common methods known in the art.

The following compounds of formula 1 according to the invention may also be obtained analogously to the Examples of synthesis described above:

### EXAMPLE 20

### 8-{2-[2-(4-fluoro-3-methoxy-phenyl)-1,1-dimethyl-ethylamino]-1-hydroxy-ethyl}-6-hydroxy-4H-benzo[1,4]oxazin-3-one;

### EXAMPLE 21

### 8-{2-[2-(4-fluoro-2,6-dimethyl-phenyl)-1,1-dimethyl-ethylamino]-1-hydroxy-ethyl}-6-hydroxy-4H-benzo[1,4]oxazin-3-one;

### EXAMPLE 22

### 8-{2-[2-(4-chloro-2-methyl-phenyl)-1,1-dimethyl-ethylamino]-1-hydroxy-ethyl}-6-hydroxy-4H-benzo[1,4]oxazin-3-one;

### EXAMPLE 23

### 8-{2-[2-(4-chloro-3-fluoro-phenyl)-1,1-dimethyl-ethylamino]-1-hydroxy-ethyl}-6-hydroxy-4H-benzo[1,4]oxazin-3-one;

### EXAMPLE 24

### 8-{2-[2-(4-chloro-2-fluoro-phenyl)-1,1-dimethyl-ethylamino]-1-hydroxy-ethyl}-6-hydroxy-4H-benzo[1,4]oxazin-3-one;

### EXAMPLE 25

### 8-{2-[2-(3-chloro-4-fluoro-phenyl)-1,1-dimethyl-ethylamino]-1-hydroxy-ethyl}-6-hydroxy-4H-benzo[1,4]oxazin-3-one;

### EXAMPLE 26

### 8-{2-[2-(2,6-difluoro-4-methoxy-phenyl)-1,1-dimethyl-ethylamino]-1-hydroxy-ethyl}-6-hydroxy-4H-benzo[1,4]oxazin-3-one;

### EXAMPLE 27

### 8-{2-[2-(2,5-difluoro-4-methoxy-phenyl)-1,1-dimethyl-ethylamino]-1-hydroxy-ethyl}-6-hydroxy-4H-benzo[1,4]oxazin-3-one;

### EXAMPLE 28

### 8-{2-[2-(4-fluoro-3,5-dimethyl-phenyl)-1,1-dimethyl-ethylamino]-1-hydroxy-ethyl}-6-hydroxy-4H-benzo[1,4]oxazin-3-one;

### EXAMPLE 29

### 8-{2-[2-(3,5-dichloro-phenyl)-1,1-dimethyl-ethylamino]-1-hydroxy-ethyl}-6-hydroxy-4H-benzo[1,4]oxazin-3-one;

### EXAMPLE 30

### 8-{2-[2-(4-chloro-3-methyl-phenyl)-1,1-dimethyl-ethylamino]-1-hydroxy-ethyl}-6-hydroxy-4H-benzo[1,4]oxazin-3-one;

### EXAMPLE 31

### 8-{2-[2-(3,4,5-trifluoro-phenyl)-1,1-dimethyl-ethylamino]-1-hydroxy-ethyl}-6-hydroxy-4H-benzo[1,4]oxazin-3-one;

### EXAMPLE 32

### 8-{2-[2-(3-methyl-phenyl)-1,1-dimethyl-ethylamino]-1-hydroxy-ethyl}-6-hydroxy-4H-benzo[1,4]oxazin-3-one;

### EXAMPLE 33

### 8-{2-[2-(3,4-dichloro-phenyl)-1,1-dimethyl-ethylamino]-1-hydroxy-ethyl}-6-hydroxy-4H-benzo[1,4]oxazin-3-one

Suitable preparations for administering the compounds of formula 1 include for example tablets, capsules, suppositories, solutions, powders, etc. The content of the pharmaceutically active compound(s) should be in the range from 0.05 to 90 wt.-%, preferably 0.1 to 50 wt.-% of the composition as a whole. Suitable tablets may be obtained, for example, by mixing the active substance(s) with known excipients, for example inert diluents such as calcium carbonate, calcium phosphate or lactose, disintegrants such as corn starch or alginic acid, binders such as starch or gelatine, lubricants such as magnesium stearate or talc and/or agents for delaying release, such as carboxymethyl cellulose, cellulose acetate phthalate, or polyvinyl acetate. The tablets may also comprise several layers.

Coated tablets may be prepared accordingly by coating cores produced analogously to the tablets with substances normally used for tablet coatings, for example collidone or shellac, gum arabic, talc, titanium dioxide or sugar. To achieve delayed release or prevent incompatibilities the core may also consist of a number of layers. Similarly the tablet coating may consist of a number of layers to achieve delayed release, possibly using the excipients mentioned above for the tablets.

Syrups or elixirs containing the active substances or combinations of active substances according to the invention may additionally contain a sweetener such as saccharine, cyclamate, glycerol or sugar and a flavour enhancer, e.g. a flavouring such as vanillin or orange extract. They may also contain suspension adjuvants or thickeners such as sodium carboxymethyl cellulose, wetting agents such as, for example, condensation products of fatty alcohols with ethylene oxide, or preservatives such as p-hydroxybenzoates.

Solutions are prepared in the usual way, e.g. with the addition of isotonic agents, preservatives such as p-hydroxybenzoates or stabilisers such as alkali metal salts of ethylenediaminetetraacetic acid, optionally using emulsifiers and/or dispersants, while if water is used as diluent, for example, organic solvents may optionally be used as solubilisers or dissolving aids, and the solutions may be transferred into injection vials or ampoules or infusion bottles.

Capsules containing one or more active substances or combinations of active substances may for example be prepared by mixing the active substances with inert carriers such as lactose or sorbitol and packing them into gelatine capsules. Suitable suppositories may be made for example by mixing with carriers provided for this purpose, such as neutral fats or polyethyleneglycol or the derivatives thereof.

Excipients which may be used include, for example, water, pharmaceutically acceptable organic solvents such as paraffins (e.g. petroleum fractions), vegetable oils (e.g. groundnut or sesame oil), mono- or polyfunctional alcohols (e.g. ethanol or glycerol), carriers such as e.g. natural mineral powders (e.g. kaolins, clays, talc, chalk), synthetic mineral powders (e.g. highly dispersed silicic acid and silicates), sugars (e.g. cane sugar, lactose and glucose), emulsifiers (e.g. lignin, spent sulphite liquors, methylcellulose, starch and polyvinylpyrrolidone) and lubricants (e.g. magnesium stearate, talc, stearic acid and sodium lauryl sulphate).

For oral use the tablets may obviously contain, in addition to the carriers specified, additives such as sodium citrate, calcium carbonate and dicalcium phosphate together with various additional substances such as starch, preferably potato starch, gelatine and the like. Lubricants such as magnesium stearate, sodium laurylsulphate and talc may also be used to produce the tablets. In the case of aqueous suspensions the active substances may be combined with various flavour enhancers or colourings in addition to the abovementioned excipients.

In the preferred use of the compounds of formula 1 for the treatment of asthma or COPD according to the invention it is particularly preferred to use preparations or pharmaceutical formulations which are suitable for inhalation. Inhalable preparations include inhalable powders, propellant-containing metered-dose aerosols or propellant-free inhalable solutions. Within the scope of the present invention, the term propellant-free inhalable solutions also includes concentrates or sterile ready-to-use inhalable solutions. The formulations which may be used within the scope of the present invention are described in more detail in the next part of the specification.

The inhalable powders which may be used according to the invention may contain 1 either on its own or in admixture with suitable physiologically acceptable excipients.

If the active substances 1 are present in admixture with physiologically acceptable excipients, the following physiologically acceptable excipients may be used to prepare these inhalable powders according to the invention: monosaccharides (e.g. glucose or arabinose), disaccharides (e.g. lactose, saccharose, maltose), oligo- and polysaccharides (e.g. dextrans), polyalcohols (e.g. sorbitol, mannitol, xylitol), salts (e.g. sodium chloride, calcium carbonate) or mixtures of these excipients. Preferably, mono- or disaccharides are used, while the use of lactose or glucose is preferred, particularly, but not exclusively, in the form of their hydrates. For the purposes of the invention, lactose is the particularly preferred excipient, while lactose monohydrate is most particularly preferred.

Within the scope of the inhalable powders according to the invention the excipients have a maximum average particle size of up to 250 μm, preferably between 10 and 150 μm, most preferably between 15 and 80 μm. In some cases it may seem appropriate to add finer excipient fractions with an average particle size of 1 to 9 μm to the excipients mentioned above. These finer excipients are also selected from the group of possible excipients listed hereinbefore. Finally, in order to prepare the inhalable powders according to the invention, micronised active substance 1, preferably with an average particle size of 0.5 to 10 μm, more preferably from 1 to 5 μm, is added to the excipient mixture. Processes for producing the inhalable powders according to the invention by grinding and micronising and lastly mixing the ingredients together are known from the prior art.

The inhalable powders according to the invention may be administered using inhalers known from the prior art.

The inhalation aerosols containing propellant gas according to the invention may contain the compounds 1 dissolved in the propellant gas or in dispersed form. The compounds 1 may be contained in separate formulations or in a common formulation, in which the compounds 1 are either both dissolved, both dispersed or in each case only one component is dissolved and the other is dispersed.

The propellant gases which may be used to prepare the inhalation aerosols are known from the prior art. Suitable propellant gases are selected from among hydrocarbons such as n-propane, n-butane or isobutane and halohydrocarbons such as fluorinated derivatives of methane, ethane, propane, butane, cyclopropane or cyclobutane. The above-mentioned propellant gases may be used on their own or mixed together. Particularly preferred propellant gases are halogenated alkane derivatives selected from TG134a and TG227 and mixtures thereof.

The propellant-driven inhalation aerosols may also contain other ingredients such as co-solvents, stabilisers, surfactants, antioxidants, lubricants and pH adjusters. All these ingredients are known in the art.

The propellant-driven inhalation aerosols according to the invention mentioned above may be administered using inhalers known in the art (MDIs=metered dose inhalers).

Moreover, the active substances 1 according to the invention may be administered in the form of propellant-free inhalable solutions and suspensions. The solvent used may be an aqueous or alcoholic, preferably an ethanolic solution. The solvent may be water on its own or a mixture of water and ethanol. The relative proportion of ethanol compared with water is not limited but the maximum is preferably up to 70 percent by volume, more particularly up to 60 percent by volume and most preferably up to 30 percent by volume. The remainder of the volume is made up of water. The solutions or suspensions containing 1 are adjusted to a pH of 2 to 7, preferably 2 to 5, using suitable acids. The pH may be adjusted using acids selected from inorganic or organic acids. Examples of particularly suitable inorganic acids include hydrochloric acid, hydrobromic acid, nitric acid, sulphuric acid and/or phosphoric acid. Examples of particularly suitable organic acids include ascorbic acid, citric acid, malic acid, tartaric acid, maleic acid, succinic acid, fumaric acid, acetic acid, formic acid and/or propionic acid etc. Preferred inorganic acids are hydrochloric and sulphuric acids. It is also possible to use the acids which have already formed an acid addition salt with one of the active substances. Of the organic acids, ascorbic acid, fumaric acid and citric acid are preferred. If desired, mixtures of the above acids may be used, particularly in the case of acids which have other properties in addition to their acidifying qualities, e.g. as flavourings, antioxidants or complexing agents, such as citric acid or ascorbic acid, for example. According to the invention, it is particularly preferred to use hydrochloric acid to adjust the pH.

If desired, the addition of editic acid (EDTA) or one of the known salts thereof, sodium edetate, as stabiliser or complexing agent may be omitted in these formulations. Other embodiments may contain this compound or these compounds. In a preferred embodiment the content based on sodium edetate is less than 100 mg/100 ml, preferably less than 50 mg/100 ml, more preferably less than 20 mg/100 ml. Generally, inhalable solutions in which the content of sodium edetate is from 0 to 10 mg/100 ml are preferred.

Co-solvents and/or other excipients may be added to the propellant-free inhalable solutions. Preferred co-solvents are those which contain hydroxyl groups or other polar groups, e.g. alcohols—particularly isopropyl alcohol, glycols—particularly propyleneglycol, polyethyleneglycol, polypropyleneglycol, glycolether, glycerol, polyoxyethylene alcohols and polyoxyethylene fatty acid esters. The terms excipients and additives in this context denote any pharmacologically acceptable substance which is not an active substance but which can be formulated with the active substance or substances in the physiologically suitable solvent in order to improve the qualitative properties of the active substance formulation. Preferably, these substances have no pharmacological effect or, in connection with the desired therapy, no appreciable or at least no undesirable pharmacological effect. The excipients and additives include, for example, surfactants such as soya lecithin, oleic acid, sorbitan esters, such as polysorbates, polyvinylpyrrolidone, other stabilisers, complexing agents, antioxidants and/or preservatives which guarantee or prolong the shelf life of the finished pharmaceutical formulation, flavourings, vitamins and/or other additives known in the art. The additives also include pharmacologically acceptable salts such as sodium chloride as isotonic agents.

The preferred excipients include antioxidants such as ascorbic acid, for example, provided that it has not already been used to adjust the pH, vitamin A, vitamin E, tocopherols and similar vitamins and provitamins occurring in the human body.

Preservatives may be used to protect the formulation from contamination with pathogens. Suitable preservatives are those which are known in the art, particularly cetyl pyridinium chloride, benzalkonium chloride or benzoic acid or benzoates such as sodium benzoate in the concentration known from the prior art. The preservatives mentioned above are preferably present in concentrations of up to 50 mg/100 ml, more preferably between 5 and 20 mg/100 ml.

Preferred formulations contain, in addition to the solvent water and the active substance 1, only benzalkonium chloride and sodium edetate. In another preferred embodiment, no sodium edetate is present.

The dosage of the compounds according to the invention is naturally highly dependent on the method of administration and the complaint which is being treated. When administered by inhalation the compounds of formula 1 are characterised by a high potency even at doses in the μg range. The compounds of formula 1 may also be used effectively above the μg range. The dosage may then be in the gram range, for example.

In another aspect the present invention relates to the above-mentioned pharmaceutical formulations as such, which are characterised in that they contain a compound of formula 1, particularly preferably the above-mentioned pharmaceutical formulations administered by inhalation.

The following examples of formulations illustrate the present invention without restricting its scope:

Examples of Pharmaceutical Formulations

| A) | Tablets | per tablet | |
|---|---|---|---|
| | active substance of formula 1 | 100 | mg |
| | lactose | 140 | mg |
| | maize starch | 240 | mg |
| | polyvinylpyrrolidone | 15 | mg |
| | magnesium stearate | 5 | mg |
| | | 500 | mg |

The finely ground active substance, lactose and some of the corn starch are mixed together. The mixture is screened, then moistened with a solution of polyvinylpyrrolidone in water, kneaded, wet-granulated and dried. The granules, the remaining corn starch and the magnesium stearate are screened and mixed together. The mixture is compressed to produce tablets of suitable shape and size.

| B) | Tablets | per tablet | |
|---|---|---|---|
| | active substance of formula 1 | 80 | mg |
| | lactose | 55 | mg |
| | maize starch | 190 | mg |
| | microcrystalline cellulose | 35 | mg |
| | polyvinylpyrrolidone | 15 | mg |
| | sodium-carboxymethyl starch | 23 | mg |
| | magnesium stearate | 2 | mg |
| | | 400 | mg |

The finely ground active substance, some of the corn starch, lactose, microcrystalline cellulose and polyvinylpyrrolidone are mixed together, the mixture is screened and worked with the remaining corn starch and water to form a granulate which is dried and screened.

The sodium carboxymethyl starch and the magnesium stearate are added and mixed in and the mixture is compressed to form tablets of a suitable size.

| C) | Ampoule solution | | |
|---|---|---|---|
| | active substance of formula 1 | 50 | mg |
| | sodium chloride | 50 | mg |
| | water for inj. | 5 | ml |

The active substance is dissolved in water at its own pH or optionally at pH 5.5 to 6.5 and sodium chloride is added to make it isotonic. The solution obtained is filtered free from pyrogens and the filtrate is transferred under aseptic conditions into ampoules which are then sterilised and sealed by fusion. The ampoules contain 5 mg, 25 mg and 50 mg of active substance.

| D) | Metered-dose aerosol | |
|---|---|---|
| | active substance of formula 1 | 0.005 |
| | sorbitolan trioleate | 0.1 |
| | monofluorotrichloromethane and TG134a:TG227 2:1 | ad 100 |

The suspension is transferred into a conventional aerosol container with a metering valve. Preferably, 50 μl of suspension are delivered per spray. The active substance may also be metered in higher doses if desired (e.g. 0.02% by weight).

| E) | Solutions (in mg/100 ml) | | |
|---|---|---|---|
| | active substance 1 | 333.3 | mg |
| | benzalkonium chloride | 10.0 | mg |
| | EDTA | 50.0 | mg |

| | |
|---|---|
| HCl (1 n) | ad pH 3.4 |

This solution may be prepared in the usual way.

| F) | Powder for inhalation | | |
|---|---|---|---|
| | active substance of formula 1 | 12 | μg |
| | lactose monohydrate | ad 10 | mg |

The powder for inhalation is produced in the usual way by mixing the individual ingredients together.

1. A method of treating respiratory complaints selected from the group comprising obstructive pulmonary diseases of various origins, pulmonary emphysema of various origins, restrictive pulmonary diseases, interstitial pulmonary diseases, cystic fibrosis, bronchitis of various origins, bronchiectasis, ARDS (adult respiratory distress syndrome) and all forms of pulmonary oedema, comprising administering to a patient in need thereof an effective amount of a compound of formula 1 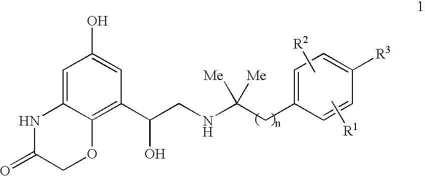
wherein
n denotes 1;
R¹ denotes hydrogen, halogen, C₁-C₄-alkyl or —O—C₁-C₄-alkyl;
R² denotes hydrogen, halogen, C₁-C₄-alkyl or —O—C₁-C₄-alkyl;
R³ denotes C₁-C₄-alkyl, OH, halogen, —O—C₁-C₄-alkyl, —O—C₁-C₄-alkylene-COOH, —O—C₁-C₄-alkylene-CO—O—C₁-C₄-alkyl,
with the proviso that if R¹ and R² each denote ortho-methyl, R³ cannot simultaneously be OH.

2) The method according to according to claim 1, wherein the compound of formula 1, wherein
n denotes 1;
R¹ denotes hydrogen, fluorine, chlorine, methyl or methoxy;
R² denotes hydrogen, fluorine, chlorine, methyl or methoxy;
R³ denotes C₁-C₄-alkyl, OH, fluorine, chlorine, bromine, —O—C₁-C₄-alkyl, —O—C₁-C₄-alkylene-COOH, —O—C₁-C₄-alkylene-CO—O—C₁-C₄-alkyl,
with the proviso that if R¹ and R² each denote ortho-methyl, R³ cannot simultaneously be OH,
is administered.

3) The method according to according to claim 1, wherein the compound of formula 1,
wherein
n denotes 1;
R¹ denotes hydrogen or C₁-C₄-alkyl;
R² denotes hydrogen or C₁-C₄-alkyl;
R³ denotes C₁-C₄-alkyl, OH, —O—C₁-C₄-alkyl, —O—C₁-C₄-alkylene-COOH or —O—C₁-C₄— alkylene-CO—O—C₁-C₄-alkyl,
with the proviso that if R¹ and R² each denote ortho-methyl, R³ cannot simultaneously be OH,
is administered.

4) The method according to according to claim 1, wherein the compound of formula 1, wherein
n denotes 1;
R¹ denotes hydrogen, methyl or ethyl;
R² denotes hydrogen, methyl or ethyl;
R³ denotes methyl, ethyl, OH, methoxy, ethoxy, —O—CH₂—COOH, —O—CH₂—COOmethyl or —O—CH₂—COOethyl,
with the proviso that if R¹ and R² each denote ortho-methyl, R³ cannot simultaneously be OH,
is administered.

5) The method according to according to claim 1, wherein the compound of formula 1, wherein
R³ denotes methoxy, ethoxy, —O—CH₂—COOH, —O—CH₂—COOmethyl or —O—CH₂—COOethyl,
n denotes 1;
R¹ denotes hydrogen; and
R² denotes hydrogen, fluorine, chlorine or methyl,
is administered.

6) The method according to according to claim 1, wherein the compound of formula 1, wherein
n denotes 1;
R¹ denotes halogen, C₁-C₄-alkyl or —O—C₁-C₄-alkyl;
R² denotes halogen, C₁-C₄-alkyl or —O—C₁-C₄-alkyl; and
R³ denotes halogen, C₁-C₄-alkyl or —O—C₁-C₄-alkyl,
is administered.

7) The method according to according to claim 6, wherein the compound of formula 1, wherein
n denotes 1;
R¹ denotes fluorine, chlorine, methyl or methoxy;
R² denotes fluorine, chlorine, methyl or methoxy; and
R³ denotes fluorine, chlorine, methyl or methoxy,
is adminsitered.

8) The method according to according to claim 1, wherein the compound of formula 1, wherein
n denotes 1;
R¹ denotes hydrogen;
R² denotes hydrogen, fluorine, chlorine or methyl; and
R³ denotes methyl, ethyl, iso-propyl, tert.-butyl, OH, fluorine, chlorine, bromine, methoxy, ethoxy, —O—CH₂—COOH, —O—CH₂—CH₂—COOH, —O—CH₂—CH₂—CH₂—COOH, —O—CH₂—COOmethyl, —O—CH₂—COOethyl, —O—CH₂—CH₂—COOmethyl, —O—CH₂—CH₂—COOethyl, —O—CH₂—CH₂—CH₂—COOmethyl, —O—CH₂—CH₂—CH₂—COOethyl,
is administered.

9) The method according to according to claim 8, wherein the compound of formula 1, wherein
n denotes 1;
R¹ denotes hydrogen;
R² denotes hydrogen, fluorine, chlorine or methyl; and
R³ denotes OH, fluorine, chlorine, methyl, methoxy, ethoxy or —O—CH₂—COOH,
is administered.

10) The method according to according to claim 1, wherein the compound of formula 1, wherein
n denotes 1;
R¹ and R² denote hydrogen; and
R³ denotes methyl, ethyl, iso-propyl, tert.-butyl, OH, fluorine, chlorine, bromine, methoxy, ethoxy, —O—CH₂—COOH, —O—CH₂—CH₂—COOH, —O—CH₂—CH₂—CH₂—COOH, —O—CH₂—COOmethyl, —O—CH₂—COOethyl, —O—CH₂—CH₂—COOmethyl, —O—CH₂—CH₂—COOethyl, —O—CH₂—CH₂—CH₂—COOmethyl, —O—CH₂—CH₂—CH₂—COOethyl,
is administered.

11) The method according to according to claim 10, wherein the compound of formula 1, wherein
n denotes 1;
R¹ and R² denote hydrogen; and
R³ denotes OH, fluorine, chlorine, methoxy, ethoxy, —O—CH₂—COOH, preferably OH, fluorine, chlorine, ethoxy or methoxy,
is administered.

12) The method according to according to claim 1, wherein the compound of formula 1, wherein
n denotes 1;
R¹ denotes hydrogen, halogen, C₁-C₄-alkyl or —O—C₁-C₄-alkyl;
R² denotes hydrogen, halogen, C₁-C₄-alkyl or —O—C₁-C₄-alkyl; and
R³ denotes hydrogen,
is administered.

13) The method according to according to claim 12, wherein the compound of formula 1, wherein
n denotes 1;
R¹ denotes hydrogen, fluorine, chlorine, methyl or methoxy;
R² denotes hydrogen, fluorine, chlorine, methyl or methoxy; and
R³ denotes hydrogen,
is administered.

14) The method of claim 1, wherein the obstructive pulmonary diseases are selected from among bronchial asthma, paediatric asthma, severe asthma, acute asthma attacks and chronic bronchitis.

15) The method of claim 1, wherein the pulmonary emphysema has its origins in COPD (chronic obstructive pulmonary disease) or α1-proteinase inhibitor deficiency.

16) The method of claim 1, wherein the restrictive pulmonary diseases are selected from among allergic alveolitis, restrictive pulmonary diseases triggered by work-related noxious substances, such as asbestosis or silicosis, and restriction caused by lung tumours selected from lymphangiosis carcinomatosa, bronchoalveolar carcinoma and lymphomas.

17) The method according to claim 1, wherein the interstitial pulmonary diseases are selected from the group consisting of pneumonia caused by infections selected from infection by viruses, bacteria, fungi, protozoa, helminths or other pathogens, pneumonitis caused by various factors selected from aspiration and left heart insufficiency, radiation-induced pneumonitis or fibrosis, collagenoses selected from lupus erythematodes, systemic sclerodermy or sarcoidosis, granulomatoses selected from Boeck's disease, idiopathic interstitial pneumonia or idiopathic pulmonary fibrosis (IPF).

18) The method according to claim 1, wherein the disease is selected from cystic fibrosis or mucoviscidosis, bronchiectasis or ARDS (adult respiratory distress syndrome).

19) The method according to claim 1, wherein the disease is bronchitis caused by bacterial or viral infection, allergic bronchitis and toxic bronchitis.

20) The method according to claim 1, wherein the disease is pulmonary oedema selected from toxic pulmonary oedema after aspiration or inhalation of toxic substances and foreign substances.
